(19) United States
(12) Patent Application Publication
(10) Pub. No.: US 2026/0283928 A1
(43) Pub. Date: Sep. 24, 2026

(54) RESIN PARTICLES AND COSMETIC

(71) Applicant: FUJIFILM Business Innovation Corp., Tokyo (JP)

(72) Inventors: Shiori CHONAN, Kanagawa (JP); Kazusei YOSHIDA, Kanagawa (JP); Ami AMANO, Kagnagawa (JP); Yuko IWADATE, Kanagawa (JP); Takahiro ISHIZUKA, Kanagawa (JP); Masahiro OKI, Kanagawa (JP); Shota MATOBA, Kanagawa (JP); Hideaki YOSHIKAWA, Kanagawa (JP); Takeshi IWANAGA, Kanagawa (JP); Sakae TAKEUCHI, Kanagawa (JP)

(73) Assignee: FUJIFILM Business Innovation Corp., Tokyo (JP)

(21) Appl. No.: 19/570,186

(22) Filed: Mar. 18, 2026

(30) Foreign Application Priority Data
Mar. 21, 2025 (JP) 2025-047485
Jan. 30, 2026 (JP) 2026-014790

(51) Int. Cl. A61K 8/73; A61Q 1/02

(52) U.S. Cl. CPC A61K 8/731; A61Q 1/02; A61K 2800/26; A61K 2800/412; A61K 2800/43; A61K 2800/522

(57) ABSTRACT

Resin particles have a transmittance at a wavelength of 550 nm of 4% or more and 20% or less in a case of being dispersed in an ester oil having a refractive index of 1.44 at 1% by mass.

### CROSS-REFERENCE TO RELATED APPLICATIONS

This application is based on and claims priority under 35 USC 119 from Japanese Patent Application No. 2026-014790 filed Jan. 30, 2026 and Japanese Patent Application No. 2025-047485 filed Mar. 21, 2025.

### BACKGROUND

### (i) Technical Field

The present invention relates to resin particles and a cosmetic.

### (ii) Related Art

JP2013-221000A proposes “spherical cellulose powder characterized in that an average primary particle diameter is 5 m or less, a primary particle diameter is in a range of 2 to 7 m for 90% by volume or more of all particles, a ratio of the longest diameter to the shortest diameter (longest diameter/shortest diameter) of a spherical shape is in a range of 1.0 to 2.5, a haze value of a 20% by mass silicone oil paste is 70% or more, and a total light transmittance is 95% or more”.

JP2022-176884A proposes “a method for improving light extraction efficiency from inside the skin, characterized by applying a cosmetic composition containing particles having a particle diameter in a Mie scattering region to the skin surface”.

### SUMMARY

Aspects of non-limiting embodiments of the present disclosure relate to resin particles capable of achieving both a shielding property in a wet state and transparency in a dry state, as compared with a case where a transmittance at a wavelength of 550 nm is less than 4% or more than 20% in a case of being dispersed in an ester oil having a refractive index of 1.44 at 1% by mass.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

Means for achieving the above object include the following aspects.

According to an aspect of the present disclosure, there are provided resin particles, in which the resin particles have a transmittance at a wavelength of 550 nm of 4% or more and 20% or less in a case of being dispersed in an ester oil having a refractive index of 1.44 at 1% by mass.

### DETAILED DESCRIPTION

Hereinafter, exemplary embodiments that are examples of the present invention will be described. The following descriptions and examples merely illustrate the exemplary embodiments, and do not limit the scope of the invention.

In a numerical range described in a stepwise manner in the present specification, an upper limit value or a lower limit value described in a certain numerical range may be substituted with an upper limit value or a lower limit value in another numerical range described in a stepwise manner. In addition, in the present specification, the upper limit or lower limit of a numerical range may be replaced with values described in Examples.

Each component may include a plurality of kinds of corresponding substances.

In a case where the amount of each component in a composition is mentioned, and there are two or more kinds of substances corresponding to each component in the composition, unless otherwise specified, the amount of each component means the total amount of two or more kinds of the substances present in the composition.

### Resin Particles

The resin particles according to the present exemplary embodiment have a transmittance at a wavelength of 550 nm of 4% or more and 20% or less in a case where the resin particles are dispersed in an ester oil having a refractive index of 1.44 at 1% by mass.

With the above-described configuration, the resin particles according to the present exemplary embodiment can achieve both a shielding property in a wet state and transparency in a dry state. The reason is presumed as follows.

The resin particles are applied to various use applications. Therefore, various functions are required for the resin particles. One of the functions thereof is a shielding property in a wet state and transparency in a dry state.

For example, in a cosmetic, which is one of the use applications of the resin particles, sweat resistance and suppression of whitening in a dry state are required to suppress makeup deterioration due to sweat. In order to impart these characteristics, the resin particles are required to have a shielding property in a wet state and transparency in a dry state.

On the other hand, the resin particles according to the present exemplary embodiment are resin particles in which the above-described transmittance is controlled. In a case where the transmittance of the resin particles is set to be in the above-described range, the light scattering of the resin particles is in an appropriate range even in a wet state, and the shielding property is ensured. In addition, since the light transmittance is in an appropriate range even in a dry state, the decrease in transparency in a dry state is suppressed.

From the above, it is presumed that the resin particles according to the present exemplary embodiment can achieve both the shielding property in a wet state and the transparency in a dry state.

Since the resin particles according to the present exemplary embodiment have the above-described characteristics, the cosmetic containing the resin particles according to the present exemplary embodiment is a cosmetic that achieves both the sweat resistance and the suppression of whitening in a dry state.

Hereinafter, the resin particles according to the present exemplary embodiment will be described in detail.

### Transmittance

In the resin particles according to the present exemplary embodiment, the transmittance at a wavelength of 550 nm in a case where the resin particles are dispersed in an ester oil having a refractive index of 1.44 at 1% by mass is 4% or more and 20% or less, for example, preferably 4% or more and 13% or less, and more preferably 4% or more and 10% or less.

In a case where the transmittance of the resin particles is less than 4%, the sense of transparency in a dry state decreases.

In a case where the transmittance of the resin particles is more than 20%, the tint is changed in a wet state, and the shielding property decreases.

In particular, in a case where the transmittance of the resin particles is within the above-described range, in a case where the resin particles are applied to a cosmetic, the cosmetic characteristics of achieving both the sweat resistance and the suppression of whitening in a dry state are improved.

The transmittance of the resin particles can be adjusted, for example, by any of the particle diameter of the resin particles, and the average major axis diameter and the porosity of the independent pores.

A method for measuring the transmittance of the resin particles is as follows.

A dispersion liquid in which resin particles to be measured are dispersed in an ester oil having a refractive index of 1.44 (specifically, hexadecyl 2-ethylhexanoate) is obtained. The amount of the resin particles in the dispersion liquid is set to 1% by mass with respect to the oil.

The obtained dispersion liquid is used as a sample, 3 mL of the sample is put into a cell having an optical path length of 10 mm, and the transmittance at a wavelength of 550 nm is measured with a spectrophotometer.

This operation is performed three times, and an arithmetic mean value of the obtained transmittances is calculated.

### Moisture Content

The resin particles according to the present exemplary embodiment preferably contain, for example, moisture. In a case where the resin particles contain moisture, the wettability is improved. In a case where resin particles having a high wetting rate are applied to a cosmetic, the moist feeling is improved.

The moisture content of the resin particles according to the present exemplary embodiment is, for example, preferably 5% by mass or more and 25% by mass or less, more preferably 7% by mass or more and 20% by mass or less, and still more preferably 11.5% by mass or more and 16% by mass or less.

In a case where the moisture content of the resin particles is 5% by mass or more, the wettability is easily improved.

In a case where the moisture content of the resin particles is 25% by mass or less, the friction coefficient of the resin particles is suppressed from being excessive.

In particular, in a case where the moisture content of the resin particles is within the above-described range, in a case where the resin particles are applied to a cosmetic, the moist feeling is improved, and the cosmetic characteristics of achieving both sweat resistance and suppression of whitening in a dry state are improved. In addition, in a case of being applied to a skin surface, the resin particles are applied to be almost uniform.

The moisture content of the resin particles can be adjusted, for example, by drying conditions during production or a surface treatment, in addition to any of the average major axis diameter or the porosity of the independent pores of the resin particles.

A method for measuring the moisture content of the resin particles is as follows.

The resin particles to be measured are left to stand for 1 day in an environment of a temperature of 25° C. and a relative humidity of 50% RH.

0.5 g of the resin particles after the standing are weighed and put into an aluminum cup, and then heated at 150° C. for 20 minutes using a heating-drying moisture meter and allowed to cool for 15 minutes. The moisture volatilized from the weight after the heating and drying is calculated.

This operation is performed three times, and an arithmetic mean value of the obtained moisture contents is calculated. The moisture content is a proportion of the volatilized moisture to the resin particles after the standing.

### Other Characteristics

In order to adjust the transmittance and the moisture content to the above-described ranges, for example, it is preferable that any one of the particle diameter, the average major axis diameter of the independent pores, or the porosity due to the independent pores of the resin particles according to the present exemplary embodiment is adjusted to a range described below.

### Volume Average Particle Diameter

The volume average particle diameter of the resin particles according to the present exemplary embodiment is, for example, preferably 1 m or more and 30 m or less, more preferably 2 m or more and 20 m or less, and still more preferably 3 m or more and 10 m or less.

In a case where the volume average particle diameter of the resin particles is within the above-described range, the porosity due to the independent pores is easily controlled to a range described below, and it is easy to achieve both the shielding property in a wet state and the transparency in a dry state.

A method for measuring the volume average particle diameter of the particles is as follows.

A particle diameter is measured by an LS particle diameter distribution measuring apparatus “Beckman Coulter LS13 320 (manufactured by Beckman Coulter Inc.)”, the cumulative distribution of the particle diameter is created from the small diameter side in terms of the volume basis, and the particle diameter that gives 50% of accumulation is determined as the volume average particle diameter.

### Average Major Axis Diameter of Independent Pores and Porosity Due to Independent Pores

The resin particles according to the present exemplary embodiment have independent pores in the particles. Here, the independent pore means a hole closed by a partition wall.

The average major axis diameter of the independent pores is, for example, preferably 0.1 m or more and 2.0 m or less, more preferably 0.1 m or more and 1.5 m or less, still more preferably 0.1 m or more and 1.0 m or less, and even more preferably 0.2 m or more and 0.8 m or less.

The porosity due to the independent pores is, for example, preferably 1% or more and 40% or less, more preferably 1% or more and 20% or less, still more preferably 1% or more and 15% or less, and particularly preferably 3% or more and 10% or less.

In a case where the average major axis diameter of the independent pores and the porosity due to the independent pores are within the above-described ranges, appropriate light scattering due to the independent pores is likely to occur, and it is easy to achieve both the shielding property in a wet state and the transparency in a dry state.

### Porosity of Communication Pores

From the viewpoint of the shielding property in a wet state, the porosity of the communication pores of the resin particles according to the present exemplary embodiment is, for example, preferably small or 0%. Here, the communication pore means a pore that is not closed by a partition wall and that leads to the outside of the resin particles (in a case where the resin particles have a coating layer described later, the outside of the base particles).

Specifically, the porosity due to the communication pores is, for example, preferably 0% or more and 10% or less, and more preferably 0% or more and 5% or less.

### Method for Confirming Independent Pores and Communication Pores, Measuring Average Major Axis Diameter of Independent Pores, and Measuring Porosity Due to Independent Pores and Communication Pores

The method for confirming the independent pores and the communication pores, the method of measuring the average major axis diameter of the independent pores, and the method of measuring the porosity due to the independent pores and the communication pores are as follows.

The resin particles are embedded in an epoxy resin, and then cut with a diamond knife or the like to produce a sample having a surface with a cross section of the resin particles as an observation surface.

The produced sample is set in a scanning electron microscope (SEM), and an SEM image (acceleration voltage: 1.0 kV, magnification: 20,000 times) of the cross section of the resin particles is obtained by imaging with the scanning electron microscope.

In the obtained SEM image, the presence of the independent pores surrounded by the partition walls and the communication pores leading from the surface of the resin particles (in a case where the resin particles have a coating layer described later, the surface of the base particles) to the inside of the particles in the cross section of the resin particles is checked.

Next, the obtained SEM image is subjected to binarization by setting a threshold value using image analysis software (for example, ImageJ, WinROOF) such that the voids and the epoxy resin, which is the background, can be clearly discriminated.

Next, the maximum diameter of the independent pores of one resin particle is measured. This operation is performed on 10 resin particles, and the average value of the maximum diameters of the independent pores is calculated as the average major axis diameter.

Next, the area ratio of all the holes with respect to the entire cross section of the resin particles is obtained. On the other hand, the area ratio of the independent pores with respect to the entire cross section of the resin particles is obtained. For the independent pores, the area ratio may be calculated without binarization as long as the independent pores can be directly identified.

In addition, the area ratio of the communication pores is calculated by the expression: area ratio of communication pores=area ratio of all pores−area ratio of independent pores. The obtained area ratio of the communication pores is defined as the porosity of the communication pores.

Then, the above-described operation is performed 10 times, and the obtained values are arithmetically averaged.

The cross section of the resin particles as the observation target is selected such that the cross-sectional diameter is 85% or more of the volume average particle diameter of the resin particles. Here, the cross-sectional diameter refers to the maximum length (so-called major axis) of a straight line drawn between any two points on the contour line of the cross section of the resin particles.

### Average Circularity

From the viewpoint of improving the shielding property in a wet state and improving the transparency in a dry state, the average circularity of the resin particles according to the present exemplary embodiment is, for example, preferably 0.88 or more, more preferably 0.90 or more, and still more preferably 0.92 or more. In particular, in a case where the resin particles are applied to cosmetic applications, for example, a high average circularity is preferable because the feeling of the skin is improved.

The average circularity of the resin particles may be, for example, ideally 1.

The circularity of the resin particles is determined by (Equivalent circle perimeter)/(Perimeter) [(Perimeter of circle having same projected area as particle image)/(Perimeter of particle image)]. Specifically, the average circularity is a value measured by the following method.

First, resin particles as a measurement target are collected by suction, and a flat flow of the particles is formed. Thereafter, an instant flash of strobe light is emitted to the particles, and the particles are imaged as a still image. By using a flow-type particle image analyzer (FPIA-3000 manufactured by Sysmex Corporation) performing image analysis on the particle image, the average circularity is determined. In addition, the number of samples in a case of determining the circularity is set to 3,500, and the arithmetic average thereof is calculated and defined as the average circularity.

### Components of Resin Particles

The resin particles according to the present exemplary embodiment contain a resin as a main component.

Here, the expression “contain a resin as a main component” means that the content of the resin with respect to the resin particles (in a case of having a coating layer described below, base particles) is 90% by mass or more (for example, preferably 95% by mass or more, 98% by mass or more, or 100% by mass).

From the viewpoint of imparting biodegradability, for example, a biodegradable resin is preferable as the resin. The biodegradable resin is a resin that is degraded into water and carbon dioxide by a microorganism. Specifically, the biodegradable resin does not have a quantitative definition of the period and the degradation rate, but for example, a resin having a biodegradation rate of 60% or more in 60 days is defined as the biodegradable resin in accordance with JIS K6950:2000 (ISO 14851:1999).

Examples of the biodegradable resin include cellulose, a cellulose derivative, a polyester resin, a natural polymer, and polyvinyl alcohol.

Among these, from the viewpoint of biodegradability, for example, cellulose or a cellulose derivative is preferable, and cellulose is more preferable.

### Cellulose

The number average molecular weight of cellulose is, for example, preferably 37,000 or more and more preferably 45,000 or more.

The upper limit value of the number average molecular weight of cellulose is not particularly limited and may be, for example, 100,000 or less.

The degree of acetyl substitution of the cellulose is, for example, preferably 0 or more and 0.1 or less, and more preferably 0 or more and 0.05 or less.

The number average molecular weight of cellulose is measured by a gel permeation chromatography method (differential refractive index meter: Optilab T-rEX, manufactured by Wyatt Technology Corporation, multi-angle light scattering detector: DAWN HELEOS II, manufactured by Wyatt Technology Corporation, TSKgel α-M column: one α-3000 column, manufactured by Tosoh Corporation) using dimethylacetamide (added with 0.1 M lithium chloride) as a solvent.

### Cellulose Derivative

Examples of the cellulose derivative include cellulose acylate, cellulose ether, hydroxyalkyl cellulose, and carboxymethyl cellulose.

Among these, as the cellulose derivative, for example, cellulose acylate is preferable. The cellulose acylate is likely to have high rollability, oil absorbency, and mechanical strength.

The cellulose acylate is a cellulose derivative in which at least some of hydroxy groups in a cellulose are substituted with acyl groups (acylated). The acyl group is a group having a structure of —CO—R^{AC} (in which R^{AC} represents a hydrogen atom or a hydrocarbon group).

The cellulose acylate is, for example, a cellulose derivative represented by General Formula (CA).

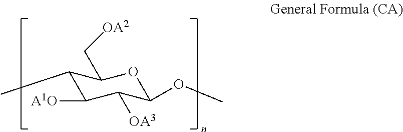

In General Formula (CA), A¹, A², and A³ each independently represent a hydrogen atom or an acyl group, and n represents an integer of 2 or more. It should be noted that at least some of n pieces of A¹'s, n pieces of A²'s, or n pieces of A³'s each represent an acyl group. The n pieces of A¹'s in the molecule may be all or partially the same as or different from each other. Similarly, n pieces of A²'s and n pieces of A³'s in the molecule may be all or partially the same as or different from each other.

In the acyl group represented by each of A¹, A², and A³, a hydrocarbon group in the acyl group may be linear, branched, or cyclic, but is, for example, preferably linear or branched, and more preferably linear.

As the acyl group represented by each of A¹, A², and A³, the hydrocarbon group in the acyl group may be a saturated hydrocarbon group or an unsaturated hydrocarbon group, but is, for example, more preferably the saturated hydrocarbon group.

The acyl group represented by each of A¹, A², and A³ is, for example, preferably an acyl group having 1 or more and 6 or less carbon atoms. That is, as the cellulose acylate, for example, cellulose acylate having an acyl group having 1 or more and 6 or less carbon atoms is preferable.

The acyl group represented by each of A¹, A², and A³ may be a group in which a hydrogen atom in the acyl group is substituted with a halogen atom (for example, a fluorine atom, a bromine atom, and an iodine atom), an oxygen atom, or a nitrogen atom, but is preferably, for example, not substituted.

Examples of the acyl group represented by each of A¹, A², and A³ include a formyl group, an acetyl group, a propionyl group, a butyryl group (butanoyl group), a propenoyl group, and a hexanoyl group. Among these, as the acyl group, for example, an acyl group having 2 or more and 4 or less carbon atoms is more preferable, and an acyl group having 2 or 3 carbon atoms is still more preferable from a viewpoint of improving the biodegradation rate of the resin particles.

Examples of the cellulose acylate include cellulose acetate (cellulose monoacetate, cellulose diacetate (DAC), or cellulose triacetate), cellulose acetate propionate (CAP), and cellulose acetate butyrate (CAB).

From the viewpoint of improving the mechanical strength, for example, cellulose acetate is preferable as the cellulose acylate.

One of cellulose acylate may be used alone, or two or more may be used in combination.

A weight average polymerization degree of cellulose acylate is, for example, preferably 200 or more and 1,000 or less, more preferably 500 or more and 1,000 or less, and further more preferably 600 or more and 1,000 or less.

The weight average polymerization degree of cellulose acylate is obtained from the weight average molecular weight (Mw) by the following procedure.

First, the weight average molecular weight (Mw) of cellulose acylate is measured by gel permeation chromatography (GPC apparatus: manufactured by Tosoh Corporation, HLC-8320GPC, column: TSKgel α-M) in terms of polystyrene using tetrahydrofuran.

Subsequently, the polymerization degree of cellulose acylate is obtained by dividing by a structural unit molecular weight of cellulose acylate. For example, in a case where a substituent of cellulose acylate is an acetyl group, the structural unit molecular weight is 263 when a substitution degree is 2.4, and 284 when the substitution degree is 2.9.

From the viewpoint of improving the rollability, the oil absorbency, and the mechanical strength, a degree of substitution of the cellulose acylate is, for example, preferably 0.75 or less, more preferably 0.6 or less, and still more preferably 0.2 or less.

The degree of substitution of cellulose acylate is an index indicating a degree to which a hydroxy group of cellulose is substituted with an acyl group. That is, the degree of substitution is an index indicating a degree of acylation of cellulose acylate. Specifically, the substitution degree means an intramolecular average number of substitutions in which three hydroxy groups in a D-glucopyranose unit of cellulose acylate are substituted with acyl groups. The degree of substitution is determined from an absorbance ratio between a peak derived from cellulose (1,030 cm⁻¹) and a peak derived from an acyl group (1,738 cm⁻¹) in an infrared absorption spectrum (Spotlight 400, manufactured by PerkinElmer, Inc.).

Specifically, a value of absorbance at 1,738 cm⁻¹/absorbance at 1,030 cm⁻¹ is defined as the degree of substitution.

### Polyester Resin

Examples of the polyester resin include an aliphatic polyester resin and an aliphatic aromatic polyester resin.

Examples of the aliphatic polyester resin include polyhydroxyalkanoic acid such as polylactic acid (PLA), polyglycolic acid (PGA), polyhydroxybutyrate, poly (3-hydroxybutyrate-co-3-hydroxyhexanoate) (PHBH), polycaprolactone, polybutylene succinate (PBS), polybutylene succinate/adipate (PBSA), and polyethylene succinate (PBA); and the like.

Examples of the aliphatic aromatic polyester resin include polybutylene adipate/terephthalate copolymer resin (PBAH), polytetramethylene adipate/terephthalate copolymer resin, and the like.

### Natural Polymer

Examples of the natural polymer include starch, cellulose, chitin, chitosan, gluten, gelatin, zein, soybean protein, collagen, and keratin.

### Other Components

The resin particles according to the present exemplary embodiment may contain other components.

Examples of the other components include a plasticizer, a flame retardant, a compatibilizer, a mold release agent, a light-resistant agent, a weather resistant agent, a colorant, a pigment, a modifier, a drip inhibitor, an antistatic agent, a hydrolysis inhibitor, a filler, a reinforcing agent (glass fiber, carbon fiber, talc, clay, mica, glass flake, milled glass, glass beads, crystalline silica, alumina, silicon nitride, aluminum nitride, boron nitride, or the like), an acid acceptor for preventing acetic acid release (an oxide such as magnesium oxide or aluminum oxide; a metal hydroxide such as magnesium hydroxide, calcium hydroxide, aluminum hydroxide, or hydrotalcite; calcium carbonate; talc; or the like), and a reactive trapping agent (for example, an epoxy compound, an acid anhydride compound, or a carbodiimide).

The content of the other components is, for example, preferably 0% by mass or more and 5% by mass or less with respect to the total amount of the resin particles (or base particles). Here, “0% by mass” means that the other components are not included.

### Layer Structure of Resin Particles

The resin particles according to the present exemplary embodiment may or may not have a coating layer.

That is, the resin particles according to the present exemplary embodiment may be resin particles having a multilayer structure including base particles containing a resin as a main component and a coating layer coating the base particles, or may be resin particles having a single-layer structure containing a resin as a main component. The base particles are particles on which the coating layer is formed.

Here, an interlayer may be provided between the base particles and the coating layer. In a case where the resin particles according to the present exemplary embodiment are, for example, resin particles having a multilayer structure including a coating layer or an interlayer, the base particles are preferably resin particles in which the transmittance at a wavelength of 550 nm is 4% or more and 20% or less in a case where 1% by mass of the base particles is dispersed in an ester oil having a refractive index of 1.44, and the coating material constituting the coating layer or the interlayer and the coating amount thereof are preferably such that the transmittance of the base particles is not changed by 2% or more due to the coating. More preferably, for example, the change in transmittance of the base particles due to the coating is less than 1%. As a result, even in a case where the coating layer is peeled off, it is possible to maintain both the shielding property in a wet state and the transparency in a dry state.

For example, in a case where the resin particles according to the present exemplary embodiment do not require water repellency, it is preferable that the resin particles do not have a coating layer, and in a case where the resin particles according to the present exemplary embodiment require water repellency, it is preferable that the resin particles have a coating layer, and it is more preferable that the resin particles have a coating layer having a coverage of 80% or more and 100% or less.

### Coating Layer

The coating layer preferably contains, for example, a coating material selected from a hydrophobic compound. Specifically, the coating layer preferably contains, for example, at least one coating material selected from the group consisting of a fatty acid, a fatty acid metal salt, and an amino acid compound.

### Fatty Acid

The fatty acid is a linear or branched saturated or unsaturated fatty acid. The fatty acid may be a mixture of a saturated fatty acid and an unsaturated fatty acid.

As the fatty acid, for example, a fatty acid having 14 or more and 22 or less carbon atoms (for example, preferably 14 or more and 20 or less carbon atoms) is preferable. Specific examples of the linear fatty acid having 14 or more and 22 or less carbon atoms include behenic acid, arachidic acid, palmitic acid, stearic acid, isostearic acid, distearic acid, and myristic acid.

### Fatty Acid Metal Salt

The fatty acid metal salt is a linear or branched saturated or unsaturated fatty acid metal salt. The fatty acid metal salt may be a mixture of a saturated fatty acid metal salt and an unsaturated fatty acid metal salt.

Examples of the fatty acid metal salt include a metal salt of a fatty acid having 14 or more and 22 or less carbon atoms (for example, preferably 14 or more and 20 or less carbon atoms). Examples of the metal salt of the fatty acid having 14 or more and 22 or less carbon atoms include a metal salt of stearic acid, a metal salt of behenic acid, a metal salt of palmitic acid, a metal salt of myristic acid, and a metal salt of distearic acid.

Examples of the metal in the fatty acid metal salt include a divalent metal.

Examples of the metal in the fatty acid metal salt include magnesium, calcium, aluminum, barium, and zinc.

### Amino Acid Compound

The amino acid compound refers to an amino acid and an amino acid derivative.

Examples of the amino acid compound include lauroyl lysine, lauryl arginine, myristyl leucine, stearoyl glutamic acid, and metal salts thereof.

The coating material constituting the coating layer is not limited to the above-described coating material, and examples thereof also include a coating material of a lipid compound (phospholipid or the like), a silicone compound (dimethylsiloxane, hydrogen dimethicone, or the like), a fluorinated compound (perfluoropolyether, perfluorooctyltriethoxysilane, or the like), and a ceramide compound (hydroxypropyl bis(palmitamide) MEA or the like).

The coating amount of the coating layer with respect to the base particles is, for example, preferably 1% by mass or more and 40% by mass or less, more preferably 3% by mass or more and 30% by mass or less, and still more preferably 10% by mass or more and 30% by mass or less.

Here, the content of the above-described coating material with respect to the entire coating layer is, for example, preferably 90% by mass or more and 100% by mass or less, and more preferably 95% by mass or more and 100% by mass or less.

The coverage of the coating layer with respect to the base particles is, for example, preferably 80% or more and 100% or less, and more preferably 90% or more and 100% or less.

In a case where the coverage of the coating layer is less than 80%, the number of regions where the base particles having high hydrophilicity are exposed increases, and the water resistance of the resin particles decreases.

A method for measuring the coverage of the coating layer is as follows.

The target resin particles are ruthenium-stained. In the scanning electron microscope observation (SEM), the coating layer is observed to be black by ruthenium staining.

The ruthenium-stained cellulose particles are observed by SEM at a magnification of 3,500 times, and a ratio of an area of the observed black coating layer to an area of one observed resin particle is calculated.

Then, this operation is performed on 50 resin particles, and an arithmetic mean value of the obtained ratios of the areas of the coating layers is calculated and defined as the coverage of the coating layer.

### Interlayer

The interlayer preferably contains, for example, at least one intermediate material selected from the group consisting of a polyamine compound, a polyquaternium, a polysaccharide compound, and a polyacrylic acid.

By providing the interlayer between the base particles and the coating layer, peeling of the coating layer is suppressed.

The polyamine compound is a general term for an aliphatic hydrocarbon having two or more primary amino groups.

Examples of the polyamine compound include polyalkyleneimine, polyallylamine, polyvinylamine, and polylysine.

From the viewpoint of improving biodegradability, the polyalkyleneimine is, for example, preferably a polyalkyleneimine having a constitutional unit having an alkylene group having 1 or more and 6 or less carbon atoms (for example, preferably 1 or more and 4 or less carbon atoms and more preferably 1 or more and 2 or less carbon atoms), and more preferably polyethyleneimine.

Examples of the polyallylamine include a homopolymer or a copolymer of allylamine, allylamine amide sulfate, diallylamine, dimethylallylamine, and the like.

The polyvinylamine is produced, for example, by hydrolyzing poly(N-vinylformamide) with an alkali, and specific examples thereof include “PVAM-0595B” manufactured by Mitsubishi Chemical Corporation.

The polylysine may be extracted from a natural product, may be produced by a transformed microorganism, or may be chemically synthesized.

Examples of the polyquaternium include polyquaternium-6, polyquaternium-7, polyquaternium-10, polyquaternium-11, polyquaternium-51, polyquaternium-61, and polyquaternium-64.

A content of the polyquaternium is, for example, preferably 0.2% by mass or more and 2% by mass or less with respect to the total amount of the base particles.

Examples of the polysaccharide compound include chitin, chitosan, and carboxymethyl cellulose. In addition, examples of the polysaccharide compound also include a polysaccharide having a structure of both of a sulfate or a phosphate and a uronic acid (for example, glucuronic acid, iduronic acid, galacturonic acid, or mannuronic acid), or a polysaccharide having a structure of both of these acids. Specific examples of the polysaccharide include hyaluronic acid, gellan gum, deacylated gellan gum (DAG), rhamsan gum, diutan gum, xanthan gum, carrageenan, zanthan gum, hexuronic acid, fucoidan, pectin, pectic acid, pectinic acid, heparan sulfate, heparin, heparitin sulfate, keratan sulfate, chondroitin sulfate, dermatan sulfate, rhamnan sulfate, and alginic acid.

As the intermediate material used for the interlayer, for example, a polysaccharide compound is preferable, and chitosan is more preferable.

A coating amount of the interlayer with respect to the base particles is, for example, preferably 0.1% by mass or more and 20% by mass or less, and more preferably 0.2% by mass or more and 2% by mass or less.

Here, a content of the intermediate material with respect to the total amount of the interlayer is, for example, preferably 90% by mass or more and 100% by mass or less, and more preferably 95% by mass or more and 100% by mass or less.

A proportion of the coating amount of the coating layer to the coating amount of the interlayer (that is, a ratio=the coating amount of the coating layer/the coating amount of the interlayer) is, for example, preferably 0.05 or more and 400 or less, more preferably 1.5 or more and 150 or less, and still more preferably 5 or more and 150 or less.

### External Additive

Inorganic particles may be added as an external additive to the resin particles according to the present exemplary embodiment. In a case where the inorganic particles are externally added, secondary aggregation of the particles is suppressed, and thus the original characteristics of the particles are likely to be exhibited.

Examples of the external additive include at least one selected from the group consisting of silicon-containing compound particles and metal oxide particles.

The silicon-containing compound particle refers to a particle containing silicon.

The silicon-containing compound particle may be a particle containing only silicon or may be a particle containing silicon and other elements.

The silicon-containing compound particle is, for example, preferably a silica particle. The silica particle may be any particle that contains silica, that is, SiO₂ as a main component, and may be crystalline or non-crystalline. In addition, the silica particle may be a particle manufactured by using a silicon compound such as water glass or an alkoxysilane as a raw material or may be a particle obtained by pulverizing quartz.

As the metal oxide, an oxide of a metal other than silicon can be applied.

Examples of the metal oxide include zinc oxide, magnesium oxide, iron oxide, and aluminum oxide.

A volume average particle diameter of the external additive is, for example, preferably 1 nm or more and 100 nm or less, and more preferably 5 nm or more and 30 nm or less.

The volume average particle diameter of the external additive is measured by the same method as the volume average particle diameter of the resin particles.

An amount of the external additive externally added is, for example, preferably 0.1% by mass or more and 2% by mass or less with respect to the total mass of the resin particles (resin particles in a state in which the external additive is not externally added).

### Method for Producing Resin Particles

A method for producing the resin particles according to the present exemplary embodiment is not particularly limited.

As a specific example, in a case of obtaining the resin particles containing cellulose as a main component, the method for producing the resin particles according to the present exemplary embodiment includes, for example, a step of granulating cellulose acylate particles (hereinafter, a granulation step) and a step of saponifying the cellulose acylate particles (hereinafter, a saponification step).

### Granulating Step

(1) First, cellulose acylate is dissolved in a water-soluble organic solvent A to prepare a cellulose acylate solution A.
(2) Next, the cellulose acylate solution A is added to a calcium carbonate dispersion liquid in which calcium carbonate and a water-soluble inorganic salt are dispersed in water, and the mixture is stirred to adjust a cellulose acylate liquid B.
(3) Next, the cellulose acylate liquid B is added to a mixed solution of carboxymethyl cellulose, the water-soluble organic solvent B, and water, and the mixture is stirred to adjust a cellulose acylate liquid C.
(4) Next, the cellulose acylate liquid C is heated to remove the water-soluble organic solvents A and B, and sodium hydroxide and hydrochloric acid are added to form cellulose acylate particles. Then, the cellulose acylate particles are separated by filtration, and the separated cellulose acylate particles are dispersed in water to prepare a cellulose acylate particle dispersion liquid D.

Here, the water-soluble organic solvents A and B are solvents in which water is dissolved in the solvent at 25° C. in an amount of 0.1% by mass or more and 10% by mass or less, and examples thereof include ethyl acetate, butyl acetate, methyl ethyl ketone, and acetone.

The water-soluble inorganic salt is, for example, preferably a salt that is dissolved in water at 20° C. in an amount of 9% or more, and particularly preferably a salt that is dissolved in water at 20° C. in an amount of 15% or more.

Examples of the water-soluble inorganic salt include sodium chloride, potassium chloride, lithium chloride, ammonium chloride, calcium chloride, magnesium chloride, sodium sulfate, potassium sulfate, ammonium sulfate, magnesium sulfate, sodium bisulfate, sodium nitrate, potassium nitrate, ammonium nitrate, calcium nitrate, magnesium nitrate, sodium carbonate, potassium carbonate, ammonium carbonate, and sodium hydrogen carbonate. For example, a sodium salt, a potassium salt, or a magnesium salt is desirable, and sodium chloride, potassium chloride, magnesium chloride, sodium sulfate, potassium sulfate, or magnesium sulfate is particularly preferable.

A concentration of the water-soluble inorganic salt in the calcium carbonate dispersion is, for example, preferably 0.5% by mass or more and 20% by mass or less, and particularly preferably 1% by mass to 10% by mass with respect to the dispersion. In a case where the concentration is too low, it is difficult to form independent pores, and in a case where the concentration is too high, it is difficult to obtain a good dispersion liquid in a case where the cellulose acylate solution A is mixed with the calcium carbonate dispersion liquid.

### Saponification Step

(5) Next, sodium hydroxide is added to the cellulose acylate particle dispersion liquid D, and then the cellulose acylate particle dispersion liquid D is heated in a weak alkaline environment and stirred to saponify the cellulose acylate particles, thereby preparing a cellulose particle suspension.
(6) Next, hydrochloric acid is added to the cellulose particle suspension to adjust the pH of the suspension to be near neutral (for example, in a range of 6.5 or more and 7 or less), and then the cellulose particles are separated by filtration and washed with an organic solvent.

Thereafter, the filtration and separation of the cellulose particles and the purified water washing are further repeated. Then, after the conductivity of the filtrate reaches 10 s/cm or less, the separated cellulose particles are dried.

Through the above-described steps, resin particles containing cellulose as a main component are obtained.

Here, in the method for producing resin particles, in a case where the cellulose acylate liquid B is added to the mixed solution of carboxymethyl cellulose, the water-soluble organic solvent B, and water, and the mixture is stirred to form oil droplets containing cellulose acylate, water droplets are contained in the oil droplets. As a result, water droplets are contained in the formed cellulose acylate particles. By drying the cellulose acylate particles, the water inside the particles evaporates, and independent pores are formed inside the obtained resin particles.

The average major axis diameter of the independent pores and the porosity due to the independent pores can be controlled by mechanical conditions such as the type of solvent, the concentration of the cellulose acylate solution A, the salt concentration of the cellulose acylate liquid B, the viscosity of the cellulose acylate liquid B, the interfacial tension between the oil phase and the water phase, and stirring in a case of adjusting the cellulose acylate liquid C.

In addition, in order to obtain resin particles having a volume average particle diameter in the above-described range, for example, the solvent species and the concentration of the cellulose acylate in the cellulose acylate solution A are adjusted.

Through the above-described operation, resin particles having a desired transmittance (and moisture content) are obtained.

In a case of obtaining resin particles containing cellulose acylate as a main component, examples thereof include a method of obtaining cellulose acylate particles in the granulation step without performing the saponification step, and a method of reducing the amount of sodium hydroxide in the saponification step to reduce the degree of saponification. By reducing the amount of sodium hydroxide in the saponification step, the degree of substitution of the cellulose acylate can be adjusted.

### Interlayer Formation Step and Coating Layer Formation Step

In a case of obtaining resin particles having a coating layer and an interlayer, the method for producing resin particles according to the present exemplary embodiment includes an interlayer formation step and a coating layer formation step after the saponification step. Specifically, the method is as follows.

First, an aqueous dispersion in which resin particles (hereinafter, “base particles”) obtained through the saponification step are dispersed is prepared. For example, it is preferable to wash the base particles with an acid before preparing the aqueous dispersion liquid.

Next, the aqueous dispersion liquid in which the base particles are dispersed and an aqueous solution containing an intermediate material constituting the interlayer are mixed. As a result, for example, the hydroxyl group of the cellulose contained in the base particles and the carboxyl group, the amino group, or the like of the intermediate material constituting the interlayer react with each other, or the hydroxyl group is hydrogen-bonded, whereby the interlayer is formed. However, in a case where the interlayer is not formed, this operation is not performed.

Then, the aqueous dispersion liquid in which the base particles on which the interlayer is formed are dispersed is heated, and then a coating material constituting the coating layer is added thereto and stirred. As a result, the coating layer is formed.

Here, in a case of forming the coating layer, the coating rate of the coating layer is controlled by the heating temperature, the addition amount of the coating material, the addition time of the coating material, and the stirring time after the addition of the coating material.

Then, the resin particles having the coating layer are taken out from the mixed solution. The extraction of the resin particles having the coating layer is performed, for example, by filtering the mixed solution. It is preferable that the extracted resin particles having the coating layer are washed with water, for example. As a result, the unreacted coating material can be removed. Then, the resin particles having the coating layer are dried to obtain the resin particles according to the present exemplary embodiment.

### External Addition Step

An external additive may be added to the obtained resin particles.

Examples of the external addition step include a treatment of adding an external additive to the cellulose particles by using a mixing mill, a V-type blender, a Henschel mixer, a Loedige mixer, or the like.

### Use Application

Examples of the use application of the resin particles according to the present exemplary embodiment include cosmetics, coating materials, rolling materials, abrasives, scrubbing agents, display spacers, beads molding materials, light diffusing particles, resin strengthening agents, refractive index control agents, biodegradation accelerators, fertilizers, water-absorbent particles, toner particles, and granules of anti-blocking particles.

### Cosmetic

The cosmetic according to the present exemplary embodiment is a cosmetic containing the resin particles according to the present exemplary embodiment, and examples thereof include the cosmetic (including a skin cleanser) described in, for example, JP2014-221743A or JP2023-150333A.

The dosage form or the form in the cosmetic according to the present exemplary embodiment is not particularly limited, and examples thereof include powder, liquid, and solid.

The cosmetic according to the present exemplary embodiment may be any of an aqueous type, an oily type, an oil-in-water type emulsion, a water-in-oil type emulsion, a non-aqueous emulsion, a W/O/W type multi-emulsion, an O/W/O type multi-emulsion, and the like.

Examples of the main preparation type of the cosmetic according to the present exemplary embodiment include a liquid type, a cream type, an aerosol, an ointment type, an emulsified solid type, a stick type, and an emulsified stick type.

The use application of the cosmetic according to the present exemplary embodiment is not particularly limited, and examples thereof include a skin care cosmetic, a hair cosmetic, a makeup cosmetic, an ultraviolet protective cosmetic, an antiperspirant, and a cleanser.

Examples of the skin care cosmetic include a skin toner, an emulsion, a cream, a cleanser, an oil liquid, a hand cream, a lip cream, a wrinkle concealing skin care cosmetic, and the like.

Examples of the hair cosmetic include a shampoo, a rinse, a treatment, a hair cream, a cuticle coat, a setting agent, and the like.

Examples of the makeup cosmetic include a makeup base, a concealer, a face powder, a powder foundation, a liquid foundation, a cream foundation, an oil foundation, a rouge, an eye shadow, a mascara, an eyeliner, an eyebrow pencil, a lipstick, a nail article, and the like.

Examples of the ultraviolet protective cosmetic include a sunblock (sunscreen) oil, a sunblock emulsion, a sunblock cream, a sunblock lotion, a sun screen agent, and the like.

In particular, since the cosmetic according to the present exemplary embodiment suppresses the coloration during the wetting with sebum and has excellent soft focus properties, for example, a makeup cosmetic is preferable.

In the cosmetic according to the present exemplary embodiment, the blending amount of the resin particles according to the present exemplary embodiment is not particularly limited, and the resin particles according to the present exemplary embodiment are appropriately blended in a range of 0.01% by mass or more and less than 100% by mass with respect to the cosmetic. The preparation type, the use application, the blending of optional components, and the blending amount of the optional components of the cosmetic are not particularly limited, and those which are known can be appropriately selected. Hereinafter, optional components will be listed. One kind of each component can be used alone, or two or more kinds of components can be used in combination.

### (1) Ultraviolet Absorber

The ultraviolet absorber is not particularly limited as long as it is a raw material that can be blended in a general cosmetic product. Specific examples thereof include oxybenzone-1 (display name (INCI: Benzophenone-1)), oxybenzone-2 (display name (INCI: Benzophenone-2)), oxybenzone-3 (display name (INCI: Benzophenone-3)), oxybenzone-4 (display name (INCI: Benzophenone-4)), oxybenzone-5 (display name (INCI: Benzophenone-5)), oxybenzone-6 (display name (INCI: Benzophenone-6)), oxybenzone-9 (display name (INCI: Benzophenone-9)), homosalate, octocrylene, t-butyl methoxydibenzoylmethane, ethylhexyl salicylate, diethylamino hydroxybenzoyl hexyl benzoate, polysilicone-15, octyl dimethoxybenzylidenedioxoimidazolidine propionate (display name (INCI: Ethylhexyldimethoxybenzylidenedioxoimidazolidine propionate)), terephthalylidenedicamphorsulfonic acid (display name (INCI: Terephthalylidenedicamphorsulfonic acid)), ethylhexyl triazone, trimethoxycinnamate methyl bis(trimethylsiloxy) isopentyl (display name (INCI: Isopentyl trimethoxycinnamate trisiloxane)), diethylhexyl dimethyl PABA (display name (INCI: Ethylhexyldimethyl PABA)), isopropyl methoxycinnamate (display name (INCI: Isopropyl methoxycinnamate)), ethylhexyl methoxycinnamate, bisethylhexyloxyphenyl methoxyphenyl triazine, phenylbenzimidazole sulfonic acid (display name (INCI: Phenylbenzimidazole sulfonic acid)), methylene bisbenzotriazolyl tetramethylbutylphenol, glyceryl ethylhexanoate dimethoxycinnamate (display name (INCI: Glyceryl ethylhexanoate dimethoxycinnamate)), glyceryl PABA, diisopropyl methyl cinnamate (display name (INCI: Diisopropyl methyl cinnamate), cinnoxate, and ethylhexyl dimethoxybenzylidenedioxoimidazolidine propionate (display name (INCI: Ethylhexyldimethoxybenzylidenedioxoimidazolidine propionate)).

### (2) Oil Agent

The oil agent may be solid, semi-solid, or liquid, and for example, natural animal and vegetable oils and fats, semi-synthetic oils, hydrocarbon oils, higher fatty acids, higher alcohols, esters, silicone oils, and fluorine-based oil agents can be used.

### Natural Animal and Vegetable Oils and Fats and Semisynthetic Fats and Oils

Examples of the natural animal and vegetable oil agent and the semi-synthetic oil agent include germ oils such as avocado oil (display name (INCI: Persea gratissima (Avocado) Oil)), linseed oil (display name (INCI: Linum usitatissimum (Linseed) Seed Oil)), sweet almond oil (display name (INCI: Prunus amygdalus dulcis (Sweet Almond) Oil)), Perilla oil, olive oil (display name (INCI: Olea europaea (Olive) Fruit Oil)), California nutmeg oil (display name (INCI: Torreya californica (California Nutmeg) Oil)), Citronella oil (display name (INCI: Cymbopogon nardus (Citronella) Oil)), Torreya Nucifera seed oil (display name (INCI: Torreya nucifera Seed Oil)), Kyounin Yu (display name (INCI: Kyounin Yu)), wheat germ oil (display name (INCI: Triticum vulgare (Wheat) Germ Oil)), sesame oil (display name (INCI: Sesamum indicum (Sesame) Seed Oil)), wheat germ oil (display name (INCI: Triticum vulgare (Wheat) Germ Oil)), rice germ oil (display name (INCI: Oryza sativa (Rice) Germ Oil)), rice bran oil (display name (INCI: Oryza sativa (Rice) Bran Oil)), sasanqua oil (display name (INCI: Camellia kissi Seed Oil)), safflower oil (display name (INCI: Carthamus tinctorius (Safflower) Seed Oil)), soybean oil (display name (INCI: Glycine soja (Soybean) Oil)), Camellia seed oil (display name (INCI: Camellia sinensis Seed Oil)), Camellia oil (display name (INCI: Camellia japonica Seed Oil)), evening primrose oil (display name (INCI: Oenothera biennis (Evening Primrose) Oil)), rapeseed oil (display name (INCI: Rape Shushi Yu)), corn germ oil (display name (INCI: Zea mays (Corn) Germ Oil)), and wheat germ oil (display name (INCI: Triticum vulgare (Wheat) Germ Oil)), natural vegetable oils such as apricot kernel oil (display name (INCI: Prunus armeniaca (APRICOT) KERNEL OIL)), palm oil (display name (INCI: Elaeis guineensis (Palm) Oil)), palm kernel oil (display name (INCI: Elaeis guineensis (Palm) Kernel Oil)), castor oil (display name (INCI: Ricinus communis (Castor) Seed Oil)), sunflower oil (display name (INCI: Helianthus annus (Sunflower) Seed Oil)), grape seed oil (display name (INCI: Vitis vinifera (Grape) Seed Oil)), jojoba seed oil (display name (INCI: Simmondsia chinensis (Jojoba) Seed Oil)), Macadamia seed oil (display name (INCI: Macadamia ternifolia Seed Oil)), meadowfoam seed oil (display name (INCI: Limnanthes Alba (Meadowfoam) Seed Oil)), cottonseed oil (display name (INCI: Gossypium herbaceum (Cotton) Seed Oil)), coconut oil (display name (INCI: Cocos nucifera (Coconut) Oil)), and peanut oil (display name (INCI: Arachis hypogaea (Peanut) Oil)), natural animal oils such as shark liver oil (display name (INCI: Shark Liver Oil)), cod liver oil (display name (INCI: Cod Liver Oil)), fish liver oil (display name (INCI: Fish Liver Oil)), turtle oil (display name (INCI: Turtle Oil)), mink oil (display name (INCI: Mink Oil)), and egg oil (display name (INCI: Egg Oil)), and semi-synthetic oils such as hydrogenated coconut oil (display name (INCI: Hydrogenated Coconut Oil) and liquid lanolin (display name (INCI: Lanolin Oil)).

### Hydrocarbon Oil

Examples thereof include an olefin oligomer, an isoparaffin such as (C13, 14) isoparaffin, isododecane, undecane, dodecane, isohexadecane, hydrogenated polyisobutene (display name (INCI: Hydrogenated Polyisobutene)), squalane (INCI), mineral oil, coconut alkane, (C13-15) alkane, and petrolatum (display name (INCI: Petrolatum).

### Higher Fatty Acid

Examples of the higher fatty acid include oleic acid (display name (INCI: Oleic Acid)), linoleic acid (display name (INCI: Linoleic Acid)), linolenic acid (display name (INCI: Linolenic Acid)), arachidonic acid (display name (INCI: Arachidonic Acid)), eicosapentaenoic acid (display name (INCI: Eicosapentaenoic Acid)), docosahexaenoic acid (display name (INCI: Docosahexaenoic Acid)), isostearic acid (display name (INCI: Isostearic Acid)), and hydroxystearic acid (display name (INCI: Hydroxystearic Acid)).

### Higher Alcohols

Examples of the higher alcohols include lauryl alcohol, myristyl alcohol, palmityl alcohol, stearyl alcohol, behenyl alcohol, oleyl alcohol, isostearyl alcohol, octyldodecanol, cholesterol, phytosterols, and batyl alcohol.

### Ester Oil

Examples of the ester oil include diisobutyl adipate (display name (INCI: Diisobutyl Adipate)), dihexyldecyl adipate (display name (INCI: NOTHING)), diheptylundecyl adipate (display name (INCI: Diheptylundecyl Adipate)), n-alkyl glycol monoisostearates such as isostearyl isostearate (display name (INCI: Isostearyl Isostearate)), isocetyl isostearate (display name (INCI: Isocetyl Isostearate)), trimethylolpropane triisostearate (display name (INCI: Trimethylolpropane Triisostearate)), glycol diethylhexanoate (display name (INCI: Glycol Diethylhexanoate)), cetyl ethylhexanoate (display name (INCI: Cetyl Ethylhexanoate)), trimethylolpropane triethylhexanoate (display name (INCI: Trimethylolpropane Triethylhexanoate)), pentaerythrityl tetraethylhexanoate (display name (INCI: Pentaerythrityl Tetraethylhexanoate)), cetyl octanoate (display name (INCI: Cetyl Ethylhexanoate)), octyldodecyl stearoyloxystearate (display name (INCI: Octyldodecyl Stearoyl Stearate)), octyldodecyl esters such as oleyl oleate (display name (INCI: Oleyl Oleate)), octyldodecyl oleate (display name (INCI: Octyldodecyl Oleate)), decyl oleate (display name (INCI: Decyl Oleate)), neopentyl glycol dioctanoate (display name (INCI: Neopentyl Glycol Diethylhexanoate)), neopentyl glycol dicaprate (display name (INCI: Neopentyl Glycol Dicaprate)), diisostearyl malate (display name (INCI: Diisostearyl Malate)), triethyl citrate (display name (INCI: Triethyl Citrate)), diethylhexyl succinate (display name (INCI: Diethylhexyl Succinate)), amyl acetate (display name (INCI: Amyl Acetate)), ethyl acetate (display name (INCI: Ethyl Acetate)), butyl acetate (display name (INCI: Butyl Acetate)), isocetyl stearate (display name (INCI: Isocetyl Stearate)), butyl stearate (display name (INCI: Butyl Stearate)), diisopropyl sebacate (display name (INCI: Diisopropyl Sebacate)), diethylhexyl sebacate (display name (INCI: Diethylhexyl Sebacate)), cetyl lactate (display name (INCI: Cetyl Lactate)), myristyl lactate (display name (INCI: Myristyl Lactate)), isononyl isononanoate (display name (INCI: Isononyl Isononanoate)), isotridecyl isononanoate (display name (INCI: Isotridecyl Isononanoate)), isopropyl palmitate (display name (INCI: Isopropyl Palmitate)), ethylhexyl isopalmitate (display name (INCI: Ethylhexyl Isopalmitate)), palmitic acid esters such as hexyldecyl palmitate (display name (INCI: Isocetyl Palmitate, Hexyldecyl Palmitate)), cholesteryl hydroxystearate (display name (INCI: Cholesteryl Hydroxystearate)), myristic acid esters such as isopropyl myristate (display name (INCI: Isopropyl Myristate)), octyldodecyl myristate (display name (INCI: Octyldodecyl Myristate)), and myristyl myristate (display name (INCI: Myristyl Myristate)), ethylhexyl laurate (display name (INCI: Ethylhexyl Laurate)), hexyl laurate (display name (INCI: Hexyl Laurate)), dioctyldodecyl lauroyl glutamate (display name (INCI: Dioctyldodecyl Lauroyl Glutamate)), and isopropyl lauroyl sarcosinate (display name (INCI: Isopropyl Lauroyl Sarcosinate).

In addition, examples of the glyceride oil in the ester oil include triethylhexanoin (INCI), tri(caprylic acid/capric acid) glyceryl (display name (INCI: Caprylic/Capric Triglyceride)), coco glyceryl (INCI), (caprylic acid/capric acid/succinic acid) triglyceride (display name (INCI: Caprylic/Capric/Succinic Triglyceride)), and (caprylic acid/capric acid) glycerides (display name (INCI: Caprylic/Capric Glycerides)).

### Silicone Oil

Examples of the silicone oil include alkyl-modified silicones such as dimethicone, trisiloxane, methyltrimethicone, ethyltrisiloxane, ethylmethicone, and hexyldimethicone; long-chain alkyl-modified silicones such as caprylyl dimethicone; linear or branched organopolysiloxanes having low viscosity to high viscosity such as phenyltrimethicone, diphenyldimethicone, diphenylsilylphenyltrimethicone, tetraphenyldimethylsiloxane, and methyl hydrogen polysiloxane; cyclic organopolysiloxanes such as cyclotetrasiloxane, cyclopentasiloxane, and cyclohexasiloxane; amino-modified organopolysiloxanes such as amodimethicone and aminopropyldimethicone; pyrrolidone-modified organopolysiloxanes such as PCA dimethicone; pyrrolidone carboxylic acid-modified organopolysiloxane; a gum-like dimethyl polysiloxane having a high degree of polymerization, a gum-like amino-modified organopolysiloxane, and a silicone rubber such as a gum-like dimethylsiloxane/methylphenylsiloxane copolymer; a low-viscosity organopolysiloxane solution of silicone gum or rubber; amino acid-modified silicone; fluorine-modified silicone; and a dissolved product of a silicone resin.

### Fluorine-Based Oil Agent

Examples of the fluorine-based oil agent include perfluorodecalin, perfluorononyl dimethicone, and perfluoromethylcyclopentane.

### (3) Compound Having Alcoholic Hydroxyl Group

Examples of the compound having an alcoholic hydroxyl group include a lower alcohol having preferably 2 to 5 carbon atoms, such as ethanol (display name (INCI: Alcohol)) and isopropanol (display name (INCI: Isopropyl Alcohol)), and a sugar alcohol such as sorbitol, maltose, and xylitol. In addition, examples thereof include sterols such as cholesterol, sitosterol (display name (INCI: Beta-Sitosterol)), phytosterols, and lanosterol.

### (4) Surfactant

Examples of the surfactant include nonionic, anionic, cationic, and amphoteric surfactants, but the surfactant is not particularly limited, and any surfactant that is used in a general cosmetic material can be used.

### (5) Powder

Examples of the powder include a colored pigment, an inorganic powder, a metal powder, an organic powder, and an inorganic-organic composite powder. Specifically, the method is as follows.

### Colored Pigment

The colored pigment is not particularly limited as long as it is a pigment that is usually used for the purpose of coloring a cosmetic material, and any colored pigment such as a composite in which different metals are doped, such as red iron oxide (display name (INCI: Iron Oxides)), yellow iron oxide (display name (INCI: Iron Oxides)), white titanium oxide (display name (INCI: Titanium Dioxide)), black iron oxide (display name (INCI: Iron Oxides)), ultramarine (display name (INCI: Ultramarines)), Prussian blue (display name (INCI: Ferric Ferrocyanide, Ferric Ammonium Ferrocyanide)), manganese violet (display name (INCI: Manganese Violet)), cobalt titanate (display name (INCI: Cobalt Titanium Oxide)), chromium hydroxide (display name (INCI: Chromium Hydroxide Green)), chromium oxide (display name (INCI: Chromium Oxide Greens)), cobalt aluminum oxide (display name (INCI: Cobalt Aluminum Oxide)), cobalt titanate (display name (INCI: Cobalt Titanium Oxide)), titanium oxide (display name (INCI: Titanium/Titanium Dioxide)), lithium cobalt titanate (display name (INCI: Lithium Cobalt Titanate)), cobalt titanate (display name (INCI: Cobalt Titanium Oxide)), (iron oxide/titanium oxide) sintered product (display name), iron oxide-doped titanium oxide (display name (INCI: Iron Oxides, Titanium Dioxide)), titanium nitride (display name (INCI: Titanium Nitride)), inorganic brown pigments such as ferrous hydroxide (display name (INCI: Iron Hydroxide)) and T-iron oxide, inorganic yellow pigments such as yellow earth, and colored pigments such as a lake of a tar-based coloring agent and a lake of a natural coloring agent can be used.

In addition, the shape of the pigment may be any shape such as a spherical shape, a substantially spherical shape, a rod shape, a spindle shape, a petal shape, a strip shape, and an irregular shape, and the geometric aspect thereof is not particularly limited as long as the cosmetic material can be colored.

### Inorganic Powder

Examples of the inorganic powder include fine particles formed of zirconium oxide (display name (INCI: Zirconium Dioxide)), zinc oxide (display name (INCI: Zinc Oxide)), cerium oxide (display name (INCI: Cerium Oxide)), magnesium oxide (display name (INCI: Magnesium Oxide)), barium sulfate (display name (INCI: Barium Sulfate)), calcium sulfate (display name (INCI: Calcium Sulfate)), magnesium sulfate (display name (INCI: Magnesium Sulfate)), calcium carbonate (display name (INCI: Calcium Carbonate)), magnesium carbonate (display name (INCI: Magnesium Carbonate)), talc, mica, kaolin, synthetic fluorophlogopite (display name (INCI: Synthetic Fluorophlogopite)), synthetic iron phlogopite, biotite (display name (INCI: Biotite)), potassium silicate (display name (INCI: Potassium Silicate)), silica, aluminum silicate (display name (INCI: Aluminum Silicate)), magnesium silicate (display name (INCI: Magnesium Silicate)), magnesium aluminum silicate (display name (INCI: Magnesium Aluminum Silicate)), calcium silicate (display name (INCI: Calcium Silicate)), aluminum calcium sodium silicate (display name (INCI: Aluminum Calcium Sodium Silicate)), lithium magnesium sodium silicate (display name (INCI: Lithium Magnesium Sodium Silicate)), sodium magnesium silicate (display name (INCI: Sodium Magnesium Silicate)), calcium aluminum borosilicate (display name (INCI: Calcium Aluminum Borosilicate)), calcium sodium borosilicate (display name (INCI: Calcium Sodium Borosilicate)), hydroxyapatite, bentonite, montmorillonite, hectorite, zeolite, aluminum, aluminum hydroxide (display name (INCI: Aluminum Hydroxide)), boron nitride (display name (INCI: Boron Nitride)), and glass (display name (INCI: Glass)).

In addition, examples of the inorganic coloring pearl pigment include pearl agents such as mica coated with titanium oxide (display name (INCI: Titanium Dioxide)) and synthetic fluorophlogopite (display name (INCI: Synthetic Fluorophlogopite)) coated with titanium oxide (display name (INCI: Titanium Dioxide)), and pearl pigments such as bismuth oxychloride (display name (INCI: Bismuth Oxychloride)), bismuth oxychloride (display name (INCI: Bismuth Oxychloride)) coated with titanium oxide (display name (INCI: Titanium Dioxide)), talc coated with titanium oxide (display name (INCI: Titanium Dioxide)), fish scale foil (display name (INCI: NOTHING)), and coloring mica coated with titanium oxide (display name (INCI: Titanium Dioxide)), and the inorganic coloring pearl pigment is not particularly limited to a known surface treatment that is generally used in cosmetics even in an untreated state.

### Metal Powder

Examples of the metal powder include metal fine particles consisting of aluminum (display name (INCI: Aluminum, Aluminum Powder)), copper (display name (INCI: Copper Powder)), silver (display name (INCI: Silver Powder)), and gold (display name (INCI: Gold)).

### Organic Powder

Examples of the organic powder include powders consisting of silicone, polyamide, polyacrylic acid acrylic acid ester, polyester, polyethylene, polypropylene, polystyrene, styrene acrylic acid copolymer, divinylbenzene styrene copolymer, polyurethane, vinyl resin, urea resin, melamine resin, benzoguanamine, polymethylbenzoguanamine, tetrafluoroethylene, polymethyl methacrylate, cellulose, silk, nylon, phenol resin, epoxy resin, and polycarbonate.

Further, examples of the organic powder also include organic pigments, and specific examples thereof include tar pigments such as Red 3, Red 104 (1) (display name (INCI: Red 28, Red 28 Lake)), Red 106, Red 201 (display name (INCI: Red 6)), Red 202 (display name (INCI: Red 7)), Red 204, Red 205, Red 220 (display name (INCI: Red 34)), Red 226 (display name (INCI: Red 30)), Red 227 (display name (INCI: Red 33, RED 33 Lake)), Red 228 (display name (INCI: Red 36)), Red 230 (1) (display name (INCI: Red 22, Red 22 Lake)), Red 230 (2) (display name), Red 401, Red 505, Yellow 4 (display name (INCI: Yellow 5)), Yellow 5 (display name (INCI: Yellow 6, Yellow 6 Lake)), Yellow 202 (1) (display name (INCI: Yellow 8)), Yellow 203 (display name (INCI: Yellow 10, Yellow 10 Lake)), Yellow 204 (display name (INCI: Yellow 11)), Yellow 401, Blue 1 (display name (INCI: Blue 1, Blue 1 Lake)), Blue 2, Blue 201, Blue 205 (display name (INCI: Blue 4)), Blue 404, Green 3 (display name (INCI: Green 3, Green 3 Lake)), Green 201 (display name (INCI: Green 5)), Green 202 (display name (INCI: Green 6)), Green 204 (display name (INCI: Green 8)), Green 205, Orange 201 (display name (INCI: Orange 5)), Orange 203 (display name (INCI: Pigment Orange 5)), Orange 204 (display name), Orange 205 (display name (INCI: Orange 4, Orange 4 Lake)), Orange 206 (display name (INCI: Orange 10)), and Orange 207 (display name (INCI: Orange 11)), and natural pigments such as cochineal, laccaic acid (display name (INCI: Lacciac Acid)), Carthamus tinctorius (Safflower) flower extract (display name (INCI: Carthamus tinctorius (Safflower) Flower Extract)), Lithospermum officinale root extract (display name (INCI: Lithospermum officinale Root Extract)), Gardenia yellow, and Gardenia blue (display name (INCI: Hydrolyzed Gardenia Florida Extract)).

### Inorganic-Organic Composite Powder

Examples of the inorganic-organic composite powder include a composite powder in which an inorganic powder surface is coated with an organic powder by a known public method. As the above-described powder, a powder in which a particle surface is treated can also be used. The treatment agent for imparting the hydrophobicity is not particularly limited, and examples thereof include a silicone treatment agent, waxes, paraffins, an organic fluorine compound such as perfluoroalkyl and a phosphate, a surfactant, an amino acid such as N-acyl glutamic acid, and a metal soap such as aluminum stearate and magnesium myristate.

### (6) Film-Forming Agent

As the film-forming agent, trimethylsiloxysilicic acid, an acrylic-silicone film-forming agent, silicone-modified norbornene, silicone-modified pullulan, or the like can be used.

### (7) Antiperspirant

The antiperspirant is not particularly limited as long as it is a component that suppresses the generation of sweat by constricting the skin, and a general-purpose component can be widely used. Examples thereof include hydroxyhalogenated aluminum such as chlorhydroxy aluminum and allantoin chlorhydroxy aluminum, aluminum halide such as aluminum chloride, allantoin aluminum salt, tannic acid, gallotannin, aluminum potassium sulfate, zinc oxide, para-phenol sulfonic acid zinc, calcined alum, tetrachloro (Al/zirconium) hydrate, trichlorohydrex glycine (Al/zirconium), and the like.

### (8) Antibacterial Agent

As the antibacterial agent, for example, antibacterial agents such as triclosan, benzalkonium chloride, benzethonium chloride, chlorhexidine hydrochloride, chlorhexidine gluconate, halocarban, and isomethyl phenol are generally used. In addition, an antibacterial agent having antibacterial properties, such as green tea dry distillation extract, a pharmaceutical herb-derived essential oil, or an extract, may be blended. As the antibacterial agent having an antibacterial effect, such as a pharmaceutical herb-derived essential oil or extract, for example, green tea extract, lavender extract, ogon extract, oren extract, obaku extract, kawarayomogi extract, kidachi aloe extract, kurara root extract, kumazasa leaf extract, garlic extract, Hamamelis extract, black tea extract, sage leaf extract, sansho extract, ginger root extract, shobu root extract, Hedera helix extract, Houttuynia cordata extract, peach fruit extract, peach leaf extract, peppermint leaf extract, senkyu extract, Eucalyptus leaf extract, peanut seed coat extract, reishi extract, and waremokou extract can be used. The plant extract may be used alone or in a mixture of two or more kinds thereof.

### (9) Other Additives

Examples of the other additives include an oil-soluble gelling agent, an ultraviolet absorbing and scattering agent, a moisturizing agent, a preservative, a fragrance, salts, an antioxidant, a pH adjusting agent, a chelating agent, a cooling agent, an anti-inflammatory agent, a skin care ingredient (a whitening agent, a cell activating agent, a skin roughness improving agent, a blood circulation promoting agent, a skin astringent, an anti-seborrheic agent, or the like), vitamins, amino acids, a nucleic acid, a hormone, and an inclusion compound.

### Oil-Soluble Gelling Agent

Examples of the oil-soluble gelling agent include metal soaps such as aluminum stearate, magnesium stearate, and zinc myristate; amino acid derivatives such as lauroyl glutamic acid (display name (INCI: Lauroyl Glutamic Acid)) and α,γ-di-n-butylamine; dextrin fatty acid esters such as dextrin palmitate (display name (INCI: Dextrin Palmitate)), dextrin isostearate (display name (INCI: Dextrin Isostearate)), dextrin myristate (display name (INCI: Dextrin Myristate)), stearoyl inulin (display name (INCI: Stearoyl Inulin)), and dextrin palmitate/ethylhexanoate (display name (INCI: Dextrin Palmitate/Ethylhexanoate)); sucrose fatty acid esters such as sucrose palmitic acid ester and sucrose stearic acid ester; fructooligosaccharide fatty acid esters such as fructooligosaccharide stearic acid ester and fructooligosaccharide 2-ethylhexanoic acid ester; benzylidene derivatives of sorbitol such as monobenzylidene sorbitol and dibenzylidene sorbitol; organically modified clay minerals of hectorite such as disteardimonium hectorite, stearalkonium hectorite, and organically modified hectorite; and stearalkonium bentonite.

### Ultraviolet Absorbing and Scattering Agent

Examples of the ultraviolet absorbing and scattering agent include particles that absorb and scatter ultraviolet rays, such as fine particle titanium oxide, fine particle iron-containing titanium oxide, fine particle zinc oxide, fine particle cerium oxide, and a composite thereof, and a dispersion in which these particles that absorb and scatter ultraviolet rays are dispersed in an oil agent in advance can also be used.

### Moisturizing Agent

Examples of the moisturizing agent include polyhydric alcohols such as BG (display name (INCI: Butylene Glycol)), PG (display name (INCI: Propylene Glycol)), DPG (display name (INCI: Dipropylene Glycol)), pentylene glycol, 1,10-decanediol, octanediol, 1,2-hexanediol, erythritol, glycerin, diglycerin, and polyethylene glycol; glucose, glyceryl glucoside, betaine, sodium chondroitin sulfate (display name (INCI: Sodium Chondroitin Sulfate)), PCA-Na (display name (INCI: Sodium PCA)), methylgluceth-10, methylgluceth-20, hyaluronic acid, egg yolk lecithin, soybean lecithin, phosphatidylcholine, phosphatidylethanolamine, phosphatidylserine, phosphatidylglycerol, phosphatidylinositol, and sphingolipid.

### Preservative

Examples of the preservative include alkyl parahydroxybenzoate, benzoic acid, sodium benzoate, sorbic acid, potassium sorbate, and phenoxyethanol, and examples of the antibacterial agent include benzoic acid, salicylic acid, phenol, sorbic acid, alkyl parahydroxybenzoate, p-chloro-m-cresol, hexachlorophene, triclocarban, a photosensitizing agent, and phenoxyethanol.

### Fragrance

The fragrance includes a natural fragrance and a synthetic fragrance. Examples of the natural fragrance include a plant-derived fragrance isolated from a flower, a leaf, a wood, a fruit peel, or the like; and an animal-derived fragrance such as musk and civet. Examples of the synthetic fragrance include hydrocarbons such as monoterpene; alcohols such as an aliphatic alcohol and an aromatic alcohol; aldehydes such as a terpene aldehyde and an aromatic aldehyde; ketones such as an alicyclic ketone; esters such as a terpene-based ester; lactones; phenols; oxides; nitrogen-containing compounds; and acetals.

### Salts

Examples of the salts include an inorganic salt, an organic acid salt, an amine salt, and an amino acid salt. Examples of the inorganic salt include a sodium salt, a potassium salt, a magnesium salt, a calcium salt, an aluminum salt, a zirconium salt, and a zinc salt of an inorganic acid such as hydrochloric acid, sulfuric acid, carbonic acid, and nitric acid; examples of the organic acid salt include a salt of an organic acid such as acetic acid, dehydroacetic acid, citric acid, malic acid, succinic acid, ascorbic acid, and stearic acid; and examples of the amine salt and the amino acid salt include a salt of an amine such as triethanolamine and a salt of an amino acid such as glutamic acid. In addition, other examples include a salt such as hyaluronic acid and chondroitin sulfate, an aluminum zirconium glycine complex, and an acid-base neutralization salt used in a cosmetic formulation.

### Antioxidant

The antioxidant is not particularly limited, and examples thereof include a carotenoid, ascorbic acid and a salt thereof, ascorbyl stearate, tocopherol, tocopheryl acetate, p-t-butylphenol, butylhydroxyanisole, dibutylhydroxytoluene, phytic acid, ferulic acid, thiotaurine, hypotaurine, a sulfite, erythorbic acid and a salt thereof, chlorogenic acid, epicatechin, epigallocatechin, epigallocatechin gallate, apigenin, kaempferol, myricetin, and quercetin. The antioxidant may be used alone or in combination of two or more kinds thereof.

### pH Adjusting Agent

Examples of the pH adjusting agent include lactic acid, citric acid, glycolic acid, succinic acid, tartaric acid, dl-malic acid, potassium carbonate, sodium hydrogen carbonate, and ammonium hydrogen carbonate.

### Chelating Agent

Examples of the chelating agent include alanine, sodium edetate, sodium polyphosphate, sodium metaphosphate, and phosphoric acid.

### Cooling Agent

Examples of the cooling agent include L-menthol and camphor.

### Anti-Inflammatory Agent

Examples of the anti-inflammatory agent include allantoin, glycyrrhizic acid and a salt thereof, glycyrrhetic acid, glycyrrhetic acid stearate, tranexamic acid, and azulene.

### Skin Care Ingredient

Examples of the skin care ingredient include a whitening agent such as placental extract, arbutin, glutathione, or a mushroom extract; a cell activator such as royal jelly, a photosensitizing agent, a cholesterol derivative, or a calf blood extract; a skin roughness improving agent; a blood circulation promoting agent such as nonyl acid vanillyl amide, benzyl nicotinate, β-butoxyethyl nicotinate, capsaicin, zingerone, cantharidin, eucommol, caffeine, tannic acid, α-bourneol, tocopherol nicotinate, inositol hexanicotinate, scillarenate, cinnarizine, trazoline, acetylcholine, verapamil, cepharanthine, or γ-oryzanol; a skin astringent such as zinc oxide or tannic acid; and an anti-seborrheic agent such as sulfur or thianthrene.

### Vitamins

Examples of the vitamins include vitamin A such as vitamin A oil, retinol, retinol acetate, or retinol palmitate; vitamin B2 such as riboflavin, riboflavin butyrate, or flavin adenine nucleotide; vitamin B6 such as pyridoxine hydrochloride, pyridoxine dioctanoate, or pyridoxin tripalmitate; vitamin B such as vitamin B12 and a derivative thereof, or vitamin B15 and a derivative thereof, vitamin C such as L-ascorbic acid, L-ascorbic acid dipalmitate ester, L-ascorbic acid-2-sodium sulfate, or L-ascorbic acid phosphoric acid diester dipotassium; vitamin D such as ergocalciferol or cholecalciferol; vitamin E such as α-tocopherol, β-tocopherol, γ-tocopherol, dl-α-tocopherol acetate, dl-α-tocopherol nicotinate, or dl-α-tocopherol succinate; nicotinic acid such as nicotinic acid, benzyl nicotinate, or nicotinamide; vitamin H; vitamin P; pantothenic acid such as calcium pantothenate, D-pantothenyl alcohol, pantothenyl ethyl ether, or acetyl pantothenyl ethyl ether; and biotin.

### Amino Acids

Examples of the amino acids include glycine, valine, leucine, isoleucine, serine, threonine, phenylalanine, arginine, lysine, aspartic acid, glutamic acid, cystine, cysteine, methionine, and tryptophan.

### Nucleic Acid

Examples of the nucleic acid include deoxyribonucleic acid.

### Hormone

Examples of the hormone include estradiol and ethinyl estradiol.

### Inclusion Compound

Examples of the inclusion compound include cyclodextrin.

### EXAMPLES

Hereinafter, Examples will be described below; however, the present invention is not limited to these Examples. Furthermore, in the following description, all “parts” and “%” are in terms of mass unless otherwise specified.

### Example 1

### Production of Resin Particles CP1

### Granulating Step

130 parts of cellulose acylate (DAC, “L-50” manufactured by Daicel Corporation) is completely dissolved in 870 parts of ethyl acetate to obtain a cellulose acylate solution. This is added to an aqueous liquid containing 90 parts of calcium carbonate and 600 parts of pure water, and the obtained solution A is stirred at a stirring speed of 8,500 rpm for 3 hours with a high-speed emulsification machine (“ULTRA-TURRAX” manufactured by IKA) to obtain a dispersion. This dispersion is added to a solution obtained by dispersing 3.2 parts of carboxymethyl cellulose (hereinafter, also referred to as “CMC”) and 120 parts of ethyl acetate in 600 parts of pure water, and the obtained dispersion B is stirred at a stirring speed of 8,500 rpm at 80° C. for 3 hours with a high-speed emulsification machine (“ULTRA-TURRAX” manufactured by IKA) to remove an organic solvent. 10 parts of sodium hydroxide and 10 parts of dilute hydrochloric acid are added thereto, the residue is filtered, and then the filtrate is dispersed in pure water again to obtain a cellulose acylate particle dispersion (concentration of solid contents: 10%).

### Saponification Step

17.5 parts of a 20% sodium hydroxide aqueous solution is added to 120 parts of the obtained cellulose acylate particle dispersion, and the mixture is stirred at a saponification temperature of 30° C. for 6 hours. Hydrochloric acid is added to the suspension after saponification to adjust the pH to 7, and the obtained cellulose particles are filtered and washed with acetone. Thereafter, the filtration and the purified water washing of the cellulose particles are repeated, and the washing is carried out until the electrical conductivity of the filtrate is 10 μs/cm or less, thereby obtaining cellulose particles.

### Surface Treatment Step

0 parts of the cellulose particles are added to ion exchange water to adjust the concentration of solid contents to 10% to obtain a slurry, 1 part of chitosan lactate (Dainichiseika Color & Chemicals Mfg. Co., Ltd.) is added thereto, and the mixture is stirred at room temperature for 5 hours, and 8 parts of sodium stearate (FUJIFILM Wako Pure Chemical Corporation) is added thereto, and the mixture is heated to 75° C. 65 parts of 5% calcium chloride is added thereto, and the mixture is stirred for 20 minutes to form a coating layer.

Thereafter, the particles are repeatedly filtered and washed, and the washing is carried out until the electrical conductivity of the filtrate is 10 μs/cm or less. After the washing, the obtained cake is dried at a temperature of 80° C. for 50 minutes using a flash jet dryer to obtain cellulose acetate particles having a coating layer.

In addition, the obtained particles are stirred with an FM mixer (FM40, manufactured by NIPPON COKE & ENGINEERING. CO., LTD.) at a rotation speed of 2,000 min⁻¹ for 3 hours while maintaining the mixer temperature at 25° C., and the surface of the coating layer is wet.

Through the above steps, resin particles CP1 are obtained.

### Example 2

Resin particles CP2 are obtained in the same manner as the resin particles CP1, except that the following items are changed.
In the granulation step, the number of parts of CMC is changed to 3.0 parts.
In the surface treatment step, the drying time is changed to 30 minutes.

### Example 3

Resin particles CP3 are obtained in the same manner as the resin particles CP1, except that the following items are changed.
In the granulation step, the number of parts of CMC is changed to 3.0 parts.
In the surface treatment step, sodium laurate is used instead of sodium stearate.

### Example 4

Resin particles CP4 are obtained in the same manner as the resin particles CP1, except that the following items are changed.
In the granulation step, the number of parts of CMC is changed to 3.3 parts.

### Example 5

Resin particles CP5 are obtained in the same manner as the resin particles CP1, except that the following items are changed.
In the granulation step, the number of parts of CMC is changed to 3.0 parts.
In the surface treatment step, the drying temperature is changed to 120° C., and the drying time is changed to 40 minutes.

### Example 6

Resin particles CP6 are obtained in the same manner as the resin particles CP1, except that the following items are changed.
In the granulation step, the number of parts of CMC is changed to 3.0 parts.
In the surface treatment step, the drying temperature is changed to 120° C., and the drying time is changed to 60 minutes.

### Example 7

Resin particles CP7 are obtained in the same manner as the resin particles CP1, except that the following items are changed.
In the granulation step, the number of parts of CMC is changed to 8.0 parts.

### Example 8

Resin particles CP8 are obtained in the same manner as the resin particles CP1, except that the following items are changed.
In the granulation step, the number of parts of CMC is changed to 1.8 parts.
In the granulation step, the stirring speed of the solution A and the dispersion liquid B is changed from 8500 rpm to 5000 rpm.

### Example 9

Resin particles CP9 are obtained in the same manner as the resin particles CP1, except that the following items are changed.
In the granulation step, the number of parts of CMC is changed to 1.5 parts.
In the granulation step, the stirring speed of the solution A and the dispersion liquid B is changed from 8500 rpm to 4000 rpm.

### Example 10

Resin particles CP10 are obtained in the same manner as the resin particles CP1, except that the following items are changed.
In the granulation step, 120 parts of ethyl acetate is changed to 200 parts of methyl ethyl ketone (MEK).

### Example 11

Resin particles CP11 are obtained in the same manner as the resin particles CP1, except that the following items are changed.
In the granulation step, 120 parts of ethyl acetate is changed to 260 parts of chloroform.

### Example 12

In the granulation step of the resin particles CP1, the cake obtained at the end of the saponification step is dried to obtain resin particles CP12 not subjected to the surface treatment.

Here, in the resin particles of Examples 1 to 11, the transmittance at a wavelength of 550 nm in a case where the base particles before the surface treatment are dispersed in an ester oil having a refractive index of 1.44 at 1% by mass is substantially the same as that of the resin particles after the surface treatment (that is, the rate of change in transmittance depending on the presence or absence of the surface treatment is less than 1%). In addition, the coverage of the coating layer with respect to the base particles is 100% in any of the resin particles.

### Comparative Example 1

Porous cellulose particles (“SELFLOW C-25” manufactured by JNR Corporation) are prepared.

### Comparative Example 2

Porous cellulose particles (“CELLULOBEADS D-10” manufactured by Daito Kasei Kogyo Co., Ltd.) are prepared.

### Comparative Example 3

Resin particles CP13 are obtained in the same manner as the resin particles CP1, except that the following items are changed.
In the saponification step, the stirring time is changed to 1 hour.

### Evaluation 1

The following evaluations are performed on the particles of Examples 1 to 5, 7 to 9, and 12, and Comparative Examples 1 to 3.

### Characteristics

Each characteristic of the particles of each example is measured according to the method described above.
Volume average particle diameter of particles (particle D50v)
Average major axis diameter of independent pores
Porosity due to independent pores
Moisture content
Transmittance at a wavelength of 550 nm in a case where the particles are dispersed in an ester oil having a refractive index of 1.44 at 1% by mass
Sweat Resistance: Shielding Property in Wet State

The bottom of a disk-shaped aluminum container (area of about 8.5 cm²) for producing a foundation is coated with red.

Next, 1 g of the resin particles of each example is placed on the bottom of the container, and a pressure of 100 kg/cm² is applied to solidify the resin particles.

Next, two drops of artificial sebum (ARTIFICIAL SEBUM, “D4265-14” manufactured by BIOCHEMAZONE) are added to the solidified solid film to wet the solid film.

Then, the degree of transparency and the degree of observation of red of the wet portion are visually observed according to the following standard.
A: The solid film is not transparentized, and red is not observed.
B: The solid film is partially transparentized, but red is not observed.
C: The solid film is transparentized, but red is not observed.
D: The solid film is transparentized, and a slight red is observed.
E: The solid film is completely transparentized, and red is observed.
Whitening: Transparency in Dry State

The monitor is asked to evaluate the whitening in a case where the resin particles are applied to the back of the hand with a finger in five stages.
A: The solid film has a glossy feeling with a sense of transparency and looks like natural bare skin.
B: The solid film has a glossy feeling and looks like natural bare skin.
C: Intermediate between B and D.
D: The solid film has a matte texture and looks slightly floating from the skin.
E: The solid film has a flat feeling and looks floating from the skin.
Moist Feeling: Wettability

The monitor is asked to evaluate the moist feeling in a case where the resin particles are applied to the back of the hand with a finger in five stages.
A: The solid film has a moist feeling and has a very good application feel.
B: The solid film has a moist feeling and has an excellent application feel.
C: The solid film has a slightly moist feeling and has no problem in the application feel.
D: The solid film has a slightly dry feeling and has a poor application feel.
E: The solid film has a dry feeling and has an extremely poor application feel.

### Evaluation 2

The resin particles of Examples 6 and 10 to 11 are evaluated in the same manner as in Evaluation 1.

TABLE 1

| | | | Porosity | Average major | | | | | | |
|---|---|---|---|---|---|---|---|---|---|---|
| | | Particle | due to | axis diameter | Moisture | | Presence | | | |
| | Resin | diameter | independent | of independent | content | | or absence | | | |
| | particles | D50v | pores | pores | (% by | Transmittance | of coating | Sweat | | Moist |
| | No. | (μm) | % | (μm) | mass) | (%) | layer | resistance | Whitening | feeling |
| Example 1 | CP 1 | 7 | 9 | 0.6 | 13 | 9 | presence | A | A | A |
| Example 2 | CP 2 | 5 | 7 | 0.5 | 12 | 6 | presence | A | B | A |
| Example 3 | CP 3 | 5 | 9 | 0.5 | 18 | 18 | presence | C | A | B |
| Example 4 | CP 4 | 6 | 7 | 0.5 | 11 | 4 | presence | A | C | B |
| Example 5 | CP 5 | 6 | 7 | 0.3 | 7 | 7 | presence | A | B | B |
| Example 6 | CP 6 | 10 | 7 | 0.3 | 5 | 10 | presence | B | A | C |
| Example 7 | CP 7 | 3 | 7 | 0.3 | 6 | 10 | presence | B | B | C |
| Example 8 | CP 8 | 28 | 7 | 0.6 | 13 | 12 | presence | B | B | B |
| Example 9 | CP 9 | 35 | 8 | 0.6 | 14 | 19 | presence | C | C | B |
| Example 10 | CP 10 | 12 | 1 | 0.3 | 7 | 13 | presence | C | B | C |
| Example 11 | CP 11 | 7 | 18 | 0.6 | 12 | 9 | presence | B | B | B |
| Example 12 | CP 12 | 7 | 9 | 0.6 | 13 | 9 | absence | A | A | B |
| Comparative | SELFLOW | 9 | 1.8 | 60 | 11 | 3.5 | absence | B | E | B |
| Example 1 | C-25 | | | | | | | | | |
| Comparative | CELLULO | 14 | 0 | 0 | 10 | 30 | absence | E | B | B |
| Example 2 | BEADS | | | | | | | | | |
| | D-10 | | | | | | | | | |
| Comparative | CP 13 | 6 | 7 | 0.3 | 25 | 25 | presence | E | B | B |
| Example 3 | | | | | | | | | | |

From the above results, it is found that the resin particles of the present examples can achieve both the sweat resistance and the suppression of whitening in a dry state, as compared with the resin particles of Comparative Examples. In addition, it is found that the resin particles of the present examples also have a moist feeling.

As a result, it is found that the resin particles of the present examples can achieve both the sweat resistance and the suppression of whitening in a dry state. In addition, it is found that the wettability is also high.

### Product Evaluation

The cosmetic evaluation is performed as follows using the resin particles CP1 of Example 1 or the resin particles CP 12 of Example 12.

### WIG Sunscreen

### Composition of WIG Sunscreen

Component 1: resin particles CP1 of Example 1: 1.0 part
Component 2: decamethylcyclopentasiloxane (Note 1): 12.0 parts
Component 3: diphenylsiloxy phenyl trimethicone (Note 2): 2.0 parts
Component 4: ethylhexyl methoxycinnamate: 7.5 parts
Component 5: diethylamino hydroxybenzoyl hexyl benzoate: 2.0 parts
Component 6: orthotriclen: 2.5 parts
Component 7: crosslinked polyether-modified silicone mixture (Note 3): 4.0 parts
Component 8: cetyl PEG/PPG-10/1 dimethicone (Note 4): 2.0 parts
Component 9: 1,3-butylene glycol: 3.0 parts
Component 10: sodium citrate: 0.5 parts
Component 11: sodium chloride: 0.5 parts
Component 12: ethanol: 5.0 parts
Component 13: purified water: 58.0 parts
(Note 1) KF-995 (manufactured by Shin-Etsu Chemical Co., Ltd.)
(Note 2) KF-56A (manufactured by Shin-Etsu Chemical Co., Ltd.)
(Note 3) KSG-210 (manufactured by Shin-Etsu Chemical Co., Ltd.)
(Note 4) KF-6048 Cetyl PEG/PPG-10/1 Dimethicone (manufactured by Shin-Etsu Chemical Co., Ltd.)

### Production and Evaluation of W/O Sunscreen

The above-described components 1 to 8 are stirred and mixed to be uniform. Separately, the components 9 to 12 are uniformly dissolved in the component 13, and the mixture is gently added to the above-described mixture of the components 1 to 8, and stirred to obtain an emulsion. The emulsion is filled in a predetermined container to obtain a W/O sunscreen.

The obtained W/O sunscreen has a light touch, does not cause an oily feeling, has good spreadability, high sense of transparency, and high water repellency, and a uniform film can be obtained.

### O/W Sunscreen Cream

### Composition of O/W Sunscreen Cream

Component 1: polyglyceryl-5 stearate (Note 1): 5.0 parts
Component 2: cetanol: 2.0 parts
Component 3: butylene glycol: 3.0 parts
Component 4: triethylhexanoin: 7.5 parts
Component 5: polyglyceryl-10 decaisostearate (Note 2): 0.5 parts
Component 6: cyclopentasiloxane: 5.0 parts
Component 7: mineral oil: 7.5 parts
Component 8: cetyl ethylhexanoate: 7.5 parts
Component 9: lipophilic fine particle titanium oxide: 5.0 parts
Component 10: lipophilic fine particle zinc oxide: 1.0 part
Component 11: resin particles CP1 of Example 1: 1.0 part
Component 12: glyceryl behenate, polyglyceryl-6 octastearate (Note 3): 0.5 parts
Component 13: xanthan gum: 0.2 parts
Component 14: bentonite (10% water-swollen liquid): 5.0 parts
Component 15: water: 49.3 parts
(Note 1) SANSOFT A-181E-C (manufactured by Taiyo Chemical Co., Ltd.)
(Note 2) SANOIL DDI (manufactured by Taiyo Chemical Co., Ltd.)
(Note 3) TAISET 50-C (manufactured by Taiyo Chemical Co., Ltd.)

### Production/Evaluation of O/W Sunscreen Cream

Operation A: component 1 and component 15 are mixed, and a part of component 2 and a part of component 3 are added thereto and mixed.
Operation B: components 4 to 8 are mixed, and components 9 to 11 and component 12 are added thereto while stirring.
Operation C: the mixture obtained in the operation B is slowly added to the mixture obtained in the operation A under stirring, and the mixture is stirred.
Operation D: the remaining component 3 and component 13 are added thereto and dispersed, and component 14 is mixed.
Operation E: the mixture obtained in the operation D is slowly added to the mixture obtained in the operation C under stirring, and the mixture is stirred to obtain an O/W sunscreen cream.

The obtained O/W sunscreen cream has a light spreadability, is not sticky and is watery, and has a uniform film and a sense of transparency.

### Lipstick

### Composition of Lipstick

Component 1: decamethylcyclopentasiloxane: 28.7 parts
Component 2: isotridecyl isononanoate: 10.0 parts
Component 3: diisostearyl malate: 5.0 parts
Component 4: candelilla wax: 10.0 parts
Component 5: polyethylene: 6.0 parts
Component 6: resin particles CP1 of Example 1: 1.0 part
Component 7: microcrystalline wax: 2.0 parts
Component 8: trimethylsiloxysilicate mixture (Note 1): 20.0 parts
Component 9: (vinyl dimethicone/lauryl dimethicone) crosspolymer (Note 2): 5.0 parts
Component 10: silicone-branched alkyl polyglyceryl co-modified silicone (Note 3): 3.0 parts
Component 11: colorant: 1.3 parts
Component 12: mica: 8.0 parts
(Note 1) Mixture of 50% of trimethylsiloxysilicate KF-7312J and 50% of cyclopentasiloxane (manufactured by Shin-Etsu Chemical Co., Ltd.)
(Note 2) Mixture of KSG-43 (vinyl dimethicone/lauryl dimethicone) crosspolymer+triethylhexanoin (manufactured by Shin-Etsu Chemical Co., Ltd.)
(Note 3) KF-6115 lauryl polyglyceryl-3 polydimethylsiloxyethyl dimethicone (manufactured by Shin-Etsu Chemical Co., Ltd.)

### Production/Evaluation of Lipstick

Operation A: A part of the component 2 is mixed with the component 11, and the obtained dispersion is heated and mixed with the component 1, the remaining part of the component 2, and the components 3 to 12 after dispersion with a roll mill to obtain a composition.
Operation B: The composition obtained in the operation A is poured into a mold, cooled, and solidified to obtain a stick-shaped lipstick.

The obtained lipstick has good spreadability and good and excellent film uniformity.

### Non-Aqueous Mousse Foundation

### Composition of Non-Aqueous Mousse Foundation

Component 1: (dimethicone/(PEG-10/15)) crosspolymer (Note 1): 18.00 parts
Component 2: dimethicone (Note 2): 1.00 part
Component 3: decamethylcyclopentasiloxane: 11.00 parts
Component 4: ethylhexyl methoxycinnamate: 5.00 parts
Component 5: jojoba oil: 1.00 part
Component 6: silylated anhydrous silicic acid (Note 3): 0.75 parts
Component 7: KTP-09R (Note 4): 0.20 parts
Component 8: KTP-09Y (Note 4): 1.00 part
Component 9: KTP-09B (Note 4): 0.02 parts
Component 10: KTP-09W (Note 4): 5.00 parts
Component 11: KF-9909 (Note 5) treated talc: 11.55 parts
Component 12: trimethylsiloxysilicic acid solution (Note 6): 4.00 parts
Component 13: decamethylcyclopentasiloxane (Note 7): 25.28 parts
Component 14: polysilicon-1 crosspolymer (Note 8): 6.00 parts
Component 15: polymethylsilsesquioxane (Note 9): 3.00 parts
Component 16: resin particles CP1 of Example 1: 7.00 parts
Component 17: antioxidant: 0.20 parts
(Note 1) Mixture of KSG-240 cyclopentasiloxane+(dimethicone/(PEG-10/15)) crosspolymer (manufactured by Shin-Etsu Chemical Co., Ltd.)
(Note 2) KF-96A (manufactured by Shin-Etsu Chemical Co., Ltd.)
(Note 3) Surface hydrophobized fumed silica: AEROSIL R-972 (manufactured by Nippon Aerosil Co., Ltd.)
(Note 4) KF-9909-treated colored inorganic pigment, W: white, R: red, Y: yellow, B: black (manufactured by Shin-Etsu Chemical Co., Ltd.)
(Note 5) Triethoxysilylethyl polydimethylsiloxyethylhexyl dimethicone (manufactured by Shin-Etsu Chemical Co., Ltd.)
(Note 6) KF-7312J trimethylsiloxy silicic acid 50% cyclopentasiloxane solution (manufactured by Shin-Etsu Chemical Co., Ltd.)
(Note 7) KF-995 (manufactured by Shin-Etsu Chemical Co., Ltd.)
(Note 8) KSP-411 (manufactured by Shin-Etsu Chemical Co., Ltd.)
(Note 9) KMP-590 (manufactured by Shin-Etsu Chemical Co., Ltd.)

### Production/Evaluation of Non-Aqueous Mousse Foundation

Components 1 to 10 are uniformly mixed by a roller treatment. Components 11 to 17 are added to this mixture and mixed to be uniform, thereby obtaining a non-aqueous mousse foundation.

The obtained foundation had a mousse-like appearance that is firmly solidified, light spreadability, and an excellent feeling of use without stickiness or oiliness, and the makeup lasting effect is also very good.

### Wash-Off Type Pack Cosmetic

### Composition of Wash-Off Type Pack Cosmetic

Component 1: dimethicone (Note 1): 3.0 parts
Component 2: decamethylcyclopentasiloxane: 3.0 parts
Component 3: polyglycerin-modified silicone oil (Note 2): 2.0 parts
Component 4: kaolin: 28.0 parts
Component 5: resin particles CP12 of Example 12: 2.0 parts
Component 6: carbomer: 0.4 parts
Component 7: butylene glycol: 10.0 parts
Component 8: glycerin: 20.0 parts
Component 9: preservative: 0.1 parts
Component 10: fragrance: 0.1 parts
Component 11: water: 31.4 parts
(Note 1) KF-96A (manufactured by Shin-Etsu Chemical Co., Ltd.)
(Note 2) KF-6105 polyglyceryl-3 polydimethylsiloxyethyl dimethicone (manufactured by Shin-Etsu Chemical Co., Ltd.)

### Production/Evaluation of Wash-Off Type Pack Cosmetic

Operation A: components 1 to 3 and component 9 are mixed.
Operation B: components 6 to 8 and component 11 are uniformly mixed, and then components 4, 5, and 10 are mixed and stirred.
Operation C: the mixture obtained in the operation A is added to the mixture obtained in the operation B, and emulsified to obtain a paste-like wash-off type pack cosmetic.

The obtained wash-off type pack cosmetic has excellent usability, which is light in spreadability during application, excellent in washing effect, and has a very excellent feeling of use after washing off, which is moist without stickiness and has a smooth feeling of the skin, and also has excellent stability.

### Deodorant Stick

### Composition of Deodorant Stick

Component 1: chlorohydroxy Al: 23.00 parts
Component 2: decamethylcyclopentasiloxane: 36.50 parts
Component 3: stearyl alcohol: 8.00 parts
Component 4: talc: 13.88 parts
Component 5: fragrance: 0.10 parts
Component 6: BHT (dibutylhydroxytoluene): 0.02 parts
Component 7: paraffin (solid): 2.00 parts
Component 8: mineral oil: 14.5 parts
Component 9: crosslinked polyether-modified silicone mixture (Note 1): 1.00 part
Component 10: resin particles CP1 of Example 1: 1.00 part
(Note 1) KF-6048 Cetyl PEG/PPG-10/1 Dimethicone (manufactured by Shin-Etsu Chemical Co., Ltd.)

### Production/Evaluation of Deodorant Stick

Operation A: components 2 to 3 and components 6 to 9 are heated and dissolved, and mixed.
Operation B: components 1, 4, and 10 are uniformly mixed with the mixture obtained in the operation A, and uniformly dispersed and mixed using a mixer.
Operation C: component 5 is added to the mixture obtained in the operation B and uniformly mixed.
Operation D: the mixture obtained in the operation C is poured into a mold and cooled and solidified.

The obtained deodorant stick had no excessive dry feeling or sticky feeling, and had excellent persistence of deodorizing effect.

### Skin Cleanser

### Composition of Skin Cleanser

Component 1: resin particles CP1 of Example 1: 1.0 part
Component 2: polyoctaninium-7 (denoted by 1): 0.1 parts
Component 3: potassium myristate: 20.0 parts
Component 4: potassium palmitate: 5.0 parts
Component 5: potassium stearate: 5.0 parts
Component 6: potassium laurate: 3.0 parts
Component 7: glycerin: 25.0 parts
Component 8: PEG-6 (H(OCH₂CH₂)ₙOH (n=average 6)): 5.0 parts
Component 9: PEG-32 (H(OCH₂CH₂)ₙOH (n=average 32)): 5.0 parts
Component 10: sorbitol: 5.0 parts
Component 11: glycosyl trehalose (denoted by 2): 2.0 parts
Component 12: glyceryl stearate: 1.5 parts
Component 13: kaolin: 1.0 part
Component 14: phytosteryl isostearate: 0.5 parts
Component 15: sodium cocoyl methyl taurate: 1.0 part
Component 16: hydrolyzed hydrogenated starch: 1.2 parts
Component 17: water: 18.7 parts
(Note 1) MERQUAT 550 (manufactured by Lubrizol Corporation)
(Note 2) TURANALE (manufactured by Hayashibara Co., Ltd.)

### Production and Evaluation of Skin Cleanser

The above-described components 1 to 17 are mixed with a homomixer to produce a skin cleanser.

A facial cleansing foam is produced from the obtained skin cleanser, and the face is washed with the facial cleansing foam. As a result, the face is thoroughly washed and has a moderate moisturizing feeling.

### Powder Foundation

### Composition of Powder Foundation

TABLE 2
Powder foundation

| | | | Part by |
|---|---|---|---|
| Formulation | Compound | Product name (manufacturer) | mass |
| Particles | Particles | Resin particles CP1 of Example 1 | 10 |
| Powders | Talc | Talc CT-25 (YAMAGUCHI MICA) | 50 |
| other than | Mica | Mica FA450 (YAMAGUCHI MICA) | 19 |
| above | Titanium oxide | MKR-1 (SAKAI CHEMICAL) | 9.5 |
| particles | Black iron oxide | C33-134 Sun CROMA Black Iron Oxide (Sun Chemical) | 0.2 |
| | Red iron oxide | C33-128 Sun CROMA Red Iron Oxide (Sun Chemical) | 0.4 |
| | Yellow iron oxide | C33-210 Sun CROMA Yellow Iron Oxide (Sun Chemical) | 2.4 |
| Bonding | Diisostearyl malate | Neosolue-dism (NIPPON FINE CHEMICAL) | 3 |
| agent | Tri(caprylic acid/capric | Tri(caprylic acid/capric acid)glyceride (FUJIFILM | 2 |
| | acid)glyceride | Wako Pure Chemical) | |
| | Neopentyl glycol dicaprate | NPDC (KOKYU ALCOHOL KOGYO) | 2 |
| | Pentylene glycol | Diol PE (KOKYU ALCOHOL KOGYO) | 1.5 |

| | |
|---|---|
| Total | 100 |

### Production and Evaluation of Powder Foundation

The components shown in Table 2 are molded by metal plate pressing to obtain a powder foundation.

The obtained powder foundation is applied to the inside of the arm with a puff, and it is smooth and has a moist feeling.

The present exemplary embodiments include the following aspects.

(((1)))

Resin particles,
wherein the resin particles have a transmittance at a wavelength of 550 nm of 4% or more and 20% or less in a case of being dispersed in an ester oil having a refractive index of 1.44 at 1% by mass.
   (((2)))

The resin particles according to (((1))),
wherein the resin particles contain a biodegradable resin as a main component.
   (((3)))

The resin particles according to (((2))),
wherein the biodegradable resin is cellulose.
   (((4)))

The resin particles according to any one of (((1))) to (((3))),
wherein the resin particles contain moisture.
   (((5)))

The resin particles according to (((4))),
wherein a moisture content is 7% by mass or more and 20% by mass or less.
   (((6)))

The resin particles according to any one of (((1))) to (((5))),
wherein a volume average particle diameter is 1 m or more and 30 m or less.
   (((7)))

The resin particles according to any one of (((1))) to (((6))),
wherein the resin particles have independent pores in the resin particles, and an average major axis diameter of the independent pores is 0.1 m or more and 2.0 m or less.
   (((8)))

The resin particles according to (((7))),
wherein a porosity due to the independent pores is 1% or more and 40% or less.
   (((9)))

The resin particles according to any one of (((1))) to (((8))),
wherein the resin particles include a base particle containing a resin as a main component, and a coating layer that coats the base particle.
   (((10)))

A cosmetic comprising:
the resin particles according to any one of (((1))) to (((9))).

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. Resin particles,
wherein the resin particles have a transmittance at a wavelength of 550 nm of 4% or more and 20% or less in a case of being dispersed in an ester oil having a refractive index of 1.44 at 1% by mass.

2. The resin particles according to claim 1,
wherein the resin particles contain a biodegradable resin as a main component.

3. The resin particles according to claim 2,
wherein the biodegradable resin is cellulose.

4. The resin particles according to claim 1,
wherein the resin particles contain moisture.

5. The resin particles according to claim 4,
wherein a moisture content is 7% by mass or more and 20% by mass or less.

6. The resin particles according to claim 1,
wherein a volume average particle diameter is 1 m or more and 30 m or less.

7. The resin particles according to claim 1,
wherein the resin particles have independent pores in the resin particles, and an average major axis diameter of the independent pores is 0.1 m or more and 2.0 m or less.

8. The resin particles according to claim 7,
wherein a porosity due to the independent pores is 1% or more and 40% or less.

9. The resin particles according to claim 1,
wherein the resin particles include a base particle containing a resin as a main component, and a coating layer that coats the base particle.

10. A cosmetic comprising:
the resin particles according to claim 1.

11. A cosmetic comprising:
the resin particles according to claim 2.

12. A cosmetic comprising:
the resin particles according to claim 3.

13. A cosmetic comprising:
the resin particles according to claim 4.

14. A cosmetic comprising:
the resin particles according to claim 5.

15. A cosmetic comprising:
the resin particles according to claim 6.

16. A cosmetic comprising:
the resin particles according to claim 7.

17. A cosmetic comprising:
the resin particles according to claim 8.

18. A cosmetic comprising:
the resin particles according to claim 9.
